(19) United States
(12) Patent Application Publication
(10) Pub. No.: US 2003/0013076 A1
(43) Pub. Date: Jan. 16, 2003

(54) Parapoxvirus vectors

(75) Inventors: Anthony J. Robinson, Canberra (AU); David J. Lyttle, Dunedin (NZ)

Correspondence Address: Nixon & Vanderhye P.C., 8th Floor, 1100 N. Glebe Rd., Arlington, VA, 22201, US

(73) Assignee: University of Otago

(21) Appl. No.: 09/796,679

(22) Filed: Mar. 2, 2001

Related U.S. Application Data
(63) Continuation of application No. 09/155,421, filed on Nov. 30, 1998, now abandoned.

(30) Foreign Application Priority Data
Mar. 29, 1996 (NZ) 286284
Mar. 27, 1997 (NZ) PCT/NZ97/00040

(51) Int. Cl. C12N 15/00; C12Q 1/70; C07H 21/04; C12N 15/09; C07K 1/00; C07K 14/00; C07K 17/00; C12N 5/00; C12N 15/74

(52) U.S. Cl. 435/5; 435/320.1; 435/325; 536/23.5; 536/23.7; 536/23.72; 530/351

(57) ABSTRACT

The invention is directed to parapoxvirus vectors. Specifically provided are orf virus vectors containing exogenous DNA. The exogenous DNA may encode a heterologous peptide or polypeptide of which expression is desired, or may encode an antigen capable of inducing an immune response. The capacity to express antigens make these vectors suitable for use in vaccines.

### TECHNICAL FIELD

This invention relates to parapoxvirus vectors, methods for their construction, and uses thereof.

### BACKGROUND OF THE INVENTION

Poxviruses are large DNA viruses which replicate within the cytoplasm of infected cells. A number of members of the poxvirus family have been used to express foreign genes. These members include vaccinia virus and avipox virus. Such viruses have the potential to deliver vaccine antigens to a variety of animal species. However, the use of modified vaccinia virus and avipox viruses are subject to a number of drawbacks.

Vaccinia virus has a wide host range in mammals. Accordingly, there is a significant risk of cross-species infection and consequent spread of disease from one species to another. This represents a significant disadvantage for any vector being used in the environment.

A further disadvantage is that vaccinia virus especially, has been shown to cause a febrile response and scarring in humans and occasionally, serious disease in an infected animal.

Avipoxviruses are more variable in their host range specificity, and while they will not generally propagate in mammals, they will often undergo an abortive infection sufficient to induce an immune response to at least some foreign genes if they are incorporated into the genome of the avipoxvirus and are expressed under control of the appropriate promoter.

Also the first infection with a vaccinia virus vector will induce an immunity to the vector such that it may limit the potential of a subsequent infection with the vector to deliver a full dose of antigen.

In the agricultural context, a major limitation to livestock production is the control of parasitic diseases. As drench resistance builds up in farmed animal populations, and consumer resistance to the use of chemical agents in livestock production also increases, there is a need for alternative means of disease control. Use of cheap, safe and effective vaccines using parapox virus vectors to deliver antigens to the host is one alternative solution which addresses these problems.

The concept of parapox virus vectors and more particularly orf virus vectors is disclosed generally by Robinson, A. J. and Lyttle, D. J. “Parapoxviruses: their biology and potential as recombinant vaccines” in Recombinant Poxviruses, Chapter 9, 306-317 eds M.Binns and G. Smith CRC Press, (1992), Boca Raton. However, there is no teaching in the reference of suitable gene insertion sites or sequences coding therefor which would allow orf virus to be used as a vector.

It is therefore an object of the present invention to provide a virus vector which goes some way toward overcoming the disadvantages outlined above in relation to existing poxvirus vectors or which at least provides the public with a useful choice.

### SUMMARY OF THE INVENTION

Accordingly, in one aspect, the present invention provides a parapoxvirus vector comprising a parapox virus containing exogenous DNA.

Preferably, the parapox virus is orf virus.

Desirably, the exogenous DNA encodes at least one gene product, and most usefully this product will be an antigen capable of inducing an immune response.

In addition, the exogenous DNA preferably further encodes at least one gene product which is a biological effector molecule, most usefully a cytokine which is capable of acting as an immunological adjuvant.

In addition, the exogenous DNA also preferably encodes a peptide moiety expressed as a hybrid or chimeric protein with a native virus protein.

Also within the scope of the invention are fragments or variants of the vector having equivalent immunological activity.

It is desirable that the exogenous DNA be incorporated in a non-essential region of the virus genome.

The exogenous DNA is preferably under the control of a poxvirus promoter, and conveniently an orf virus promoter.

In a further aspect, the present invention provides a method for the production of parapoxvirus vectors, replicable transfer vectors for use in the method of the invention and hosts transformed with these vectors.

In a further aspect the invention consists in a vaccine which includes a parapoxvirus vector defined above in combination with a pharmaceutically acceptable carrier and optionally or alternatively, an adjuvant therefor.

In a still further aspect the present invention relates to the use of parapoxvirus vectors to prepare heterologous polypeptides in eukaryotic cells comprising infecting cells with the parapoxvirus vector and isolating the heterologous polypeptide once expressed.

Although the invention is broadly as described above, it will be appreciated by those persons skilled in the art that the invention is not limited to the foregoing but also includes embodiments of which the following gives examples. In particular, certain aspects of the invention will be more clearly understood by having reference to the accompanying drawings.

### BRIEF DESCRIPTION OF THE DRAWINGS

FIG. 1 represents a map of the genomes of the orf virus strains NZ-2, NZ-7 and NZ-10 showing cleavage sites for the restriction endonuclease KpnI. The genomes are double stranded DNA molecules and are represented as horizontal lines. The positions of the endonuclease cleavage sites on each genome relative to the ends of the genome are represented by vertical lines. Individual genome fragments that would be generated by digestion with the endonuclease are designated with letters of the alphabet.

FIG. 2 represents a nucleotide sequence of a region of the KpnI E fragment of the orf virus strain NZ-2 genome. The sequence underlined with a dashed line contains potential insertion sites. The sequence underlined with colons represents that portion of a vascular endothelial growth factor like gene that contains potential insertion sites.

FIG. 3 represents a nucleotide sequence of a region of the KpnI D fragment of the orf virus strain NZ-7 genome in FIG. 1. The sequences underlined with a dashed line represent sites for the insertion of foreign genes. The sequence underlined with colons represents that portion of a vascular endothelial growth factor-like gene that contains potential insertion sites.

FIG. 4 represents a map of the genome of the orf virus strain NZ-2 showing cleavage sites for the restriction endonuclease HindIII. The genome is a double stranded DNA molecule and is here represented as a horizontal line. The positions of the endonuclease cleavage sites on the genome relative to the ends of the genome are represented by vertical lines. Individual genome fragments that would be generated by digestion with the endonuclease are designated with letters of the alphabet. The region comprising part of fragment F, all of fragments J and I and part of fragment E for which the DNA sequence has been determined is shown. Open reading frames encoding putative genes are shown. The open reading frames encoding the putative genes (H)I1L and (H)I2L contain potential insertion sites. In addition the intergenic regions between rpo132 and (H)I1L, (H)I1L and (H)I2L, (H)I2L and (H)E1L and (H)E1L and (H)E2L represent potential insertion sites.

FIG. 5 represents the nucleotide sequence of the open reading frames depicted in FIG. 4. The genes (H)I1L, and (H)I2L which contain potential insertion sites are underlined with colons. Potential insertion sites within intergenic regions are underlined with a dotted line. Putative promoter sequences are marked by asterisks.

FIG. 6 represents a map of the genome of the orf virus strain NZ-2 showing cleavage sites for the restriction endonuclease BamHI The genome is a double stranded DNA molecule and is here represented as a horizontal line. The positions of the endonuclease cleavage sites on the genome relative to the ends of the genome are represented by vertical lines. Individual genome fragments that would be generated by digestion with the endonuclease are designated with letters of the alphabet. The region comprising fragment BamHI F and part of BamHI C for which the DNA sequence has been determined is shown. Open reading frames encoding DNA topoisomerase (F4R) and the putative genes F1L, F2L, F3R and C1L are shown as unfilled arrows.

FIG. 7 represents a nucleotide sequence of the BamHI F fragment and part of the BamHI C fragment of the orf virus strain NZ-2 genome shown in FIG. 6. The sequences underlined with a dashed line represent potential insertion sites. The putative promoter sequences PF1L, PF2L, PF3R, PF4R and PC1R are marked by asterisks.

FIG. 8 represents a map of the genome of orf virus strain NZ-2 showing cleavage sites for the restriction endonuclease BamHI. The genome is a double stranded DNA molecule and is here represented as a horizontal line. The positions of the endonuclease cleavage sites on the genome relative to the ends of the genome are represented by vertical lines. Individual genome fragments that would be generated by digestion with the endonuclease are designated with letters of the alphabet. The region comprising fragments BamHI H, BamHI E, BamHI G and part of BamHI B for which the DNA sequence has been determined is shown. Open reading frames encoding putative genes are shown as unfilled arrows. The position of a 3.3 kilobase pair deletion encompassing open reading frames E2L, E3L and G1L is shown.

FIG. 9 represents a nucleotide sequence of a region of the BamHI E fragment and BamHI G fragment of the orf virus strain NZ-2 genome shown in FIG. 8. Potential insertion sites underlined by colons are present in the region which encodes for the putative genes E2L, E3L and G1L. Potential insertion sites within intergenic regions are underlined with a dotted line. Putative promoter sequences are marked by asterisks. The region located between the ITR junction and the marked endpoint of deletion is absent in a variant strain derived from NZ-2.

FIG. 10 represents nucleotide sequences from the orf virus genome strain NZ-2 that act as transcriptional promoters. Early and late promoter sequences are indicated. For each sequence the left hand end is the 5′ end.

FIG. 11 is a diagram representing the steps in the construction of the plasmid pSP-PFlac.

FIG. 12 is a diagram representing the steps in the construction of the plasmid pSP-SFPgpt32.

FIG. 13 is a diagram representing the steps in the construction of the plasmid pFS-gpt.

FIG. 14 is a diagram representing the steps in the construction of the plasmids pVU-DL104 and pVU-DL106.

FIG. 15 is a diagram representing the steps in the construction of the plasmids ptov2 and ptov3.

FIG. 16 is a diagram representing the steps in the construction of the plasmid ptov6.

FIG. 17 is a diagram representing the steps in the construction of the plasmid ptov8.

FIG. 18 is a diagram representing the steps in the construction of the plasmids pVU-DL45W and pVU-DL45W1.

FIG. 19 is a diagram representing the steps in the construction of the plasmids pVU-DL45Wlac and pVU-DL45W1lac.

FIG. 20 outlines a strategy for the generation of recombinant orf virus.

FIG. 21A provides the nucleic acid sequence for the primers zxs-1, zxs-2, zxs-3 and zxs-4 used for the amplification of orf virus sequences used to create the transfer vector pTvec50.

FIG. 21B provides the nucleic acid sequence for the modified intergenic region between the RNA polymerase subunit gene, rpo 132, and (H)I1L in pTvec50, showing new created restriction sites for the restriction enzymes ApoI, NsiI, NcoI and EcoRI. The priming sites on the original OV sequence for the zxs-3 primer are marked by asterisks, the newly created transcriptional termination signal (TTTTTAT) is shown in bold type.

FIG. 22 is a diagram representing the steps in the construction of the plasmids pTvec1 and pTvec-50.

FIG. 23 is a diagram representing the steps in the construction of the transfer vectors pTvec50lac-1 and pTvec50lac-2.

In a first aspect the present invention provides a parapoxvirus vector comprising a parapox virus containing exogenous DNA. Preferably, the parapoxvirus is an orf virus. Orf virus has a relatively narrow host range being generally confined to sheep, goats, monkeys and man. The narrow host range avoids the disadvantage associated with the use of vaccinia virus as a vector in the environment. In particular, cross-species infection will be limited. Most animals and birds would simply undergo an abortive infection of the orf virus, but the orf virus may still be capable of delivering an immunising dose of some antigens.

Accordingly, the narrow host range may allow the use of orf virus in animals normally resistant to infection with orf virus to stimulate an immune response. The orf virus may also be particularly useful in delivering antigens to birds, where the virus does not propagate in avian species.

Orf virus also has the advantage of being less virulent than vaccinia virus in man. Unlike vaccinia virus, orf virus does not cause a febrile response and lesions are shown to heal without scarring. Ideally the orf virus vector will lack its original virulence factor. Orf virus is reviewed in Robinson, A. J. and Balassu, T. C. (1981) Contagious pustular dermatitis (orf). Vet Bull 51 771-761 and Robinson, A. J. and Lyttle, D. J. (1992) “Parapoxviruses: their biology and potential as recombinant vaccines” in Recombinant Poxviruses, Chapter 9, 306-317 eds M.Binns and G. Smith CRC Press, (1992), Boca Raton.

The term “containing exogenous DNA” as used herein refers to exogenous DNA which is incorporated into the virus genome.

Preferably, the exogenous DNA in the orf virus vector is a gene encoding a gene product or products. The gene product may be a heterologous peptide or polypeptide but most usefully, the gene product is an antigen or antigens capable of eliciting an immune response in an infected host. Exogenous DNA encoding genes for a combination of antigens is also possible. The antigen(s) may also be treated with suitable inhibitors, modifiers, crosslinkers and/or denaturants to enhance its stability or immunogenicity if required.

Some examples of foreign genes of medical and veterinary importance which may potentially be incorporated into orf virus include HIV envelope protein, herpes simplex virus glycoprotein, Taenia ovis antigens, Echinococcus granulosus (hydatids) antigens, Trichostrongylus and antigens of gastrointestinal parasites such as Haemonchus and Ostertagia or combinations thereon but are not limited thereto.

Preferred antigens include Taenia ovis 45W, 16 kd and 18 kd antigens as disclosed in WO 94/22913 incorporated herein by reference.

In a further preferred embodiment, the exogenous DNA may further comprise a cytokine gene or genes coding for other biological effector molecules which modify or augment an immune response, in combination with the exogenous antigenic DNA. Preferred cytokine genes include γ interferon and the interleukins comprising IL-1, IL-2, IL-1β, IL-4, IL-5, IL-6, IL-12 and most preferably IL-1, IL-2 and IL-12 either alone or in combination.

In another embodiment the exogenous DNA may further comprise one or more reporter genes and/or at least one gene coding for a selectable marker.

Examples of suitable well known reporter genes include Escherichia coli β-galactosidase (lacz), Photinus pyralis firefly luciferase (lux), secreted placental alkaline phosphatase (SEAP) and Aequorea victoria green fluorescent protein (gfp).

Selectable marker genes known and suitable for use in the present invention include xanthine-guanine phosphoribosyl transferase gene (xgpt), and neomycin phosphotransferase (aphII)

In a particularly preferred embodiment the exogenous DNA will comprise genes encoding multiple antigens in combination with one or more biological effector DNA molecules to enhance immune response. In practical terms where multiple antigens are coded for they will generally number 20 or less, preferably 10 or less.

Additionally, the DNA preferably encodes a peptide moiety expressed as a hybrid or chimeric protein with a native virus protein.

In this embodiment of the invention the exogenous DNA encodes for a peptide sequence that forms part of a virus protein. The native protein would retain its original properties but would exhibit additional antigenic epitopes, enzymatic properties or receptor-binding functions encoded by the exogenous DNA. Such a chimeric protein could be secreted, or could form part of the virus envelope or could form part of the virus capsid.

Also within the scope of the invention are fragments or variants of a vector of the invention having equivalent immunological activity. Such variants may be produced by the insertion, deletion or substitution of one or more amino acids using techniques known in the art (Sambrook, J. Fritsch, E. F. and Maniatis, T. Molecular Cloning, A Laboratory Manual (Second Edition) Cold Spring Harbour Laboratory Press 1989).

As will be appreciated by the reader, it is also desirable for the foreign gene to be incorporated into a non-essential region of the orf virus genome. In particular, the gene must be inserted into a region where it does not disrupt viral replication.

Surprisingly, the non-essential thymidine kinase gene, which is used as an insertion site in vaccinia virus has not been found in orf virus. It was therefore necessary to identify alternative non-essential sites in orf virus.

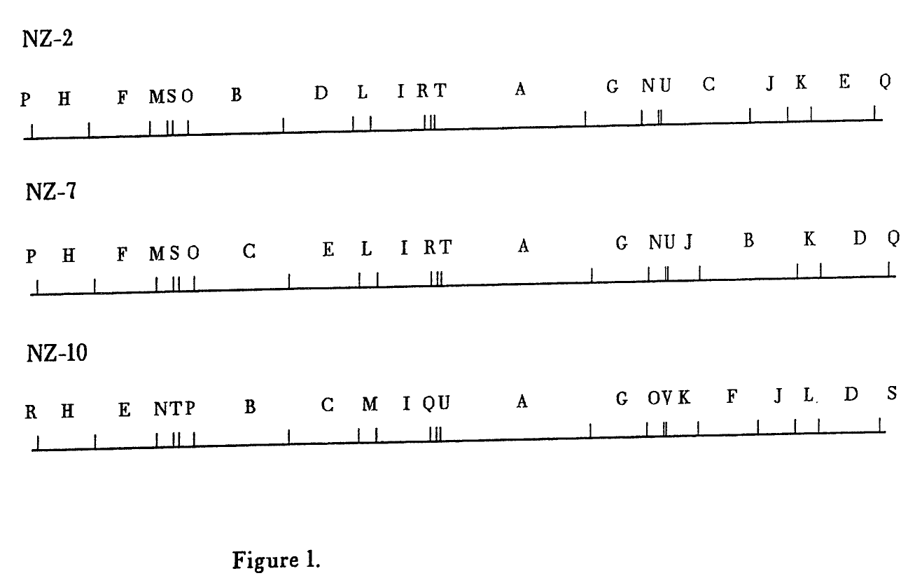

Non-essential sites were identified following restriction enzyme mapping of orf virus DNA. DNA maps for orf virus stains NZ-2, NZ-7 and NZ-10 are shown in accompanying FIG. 1.

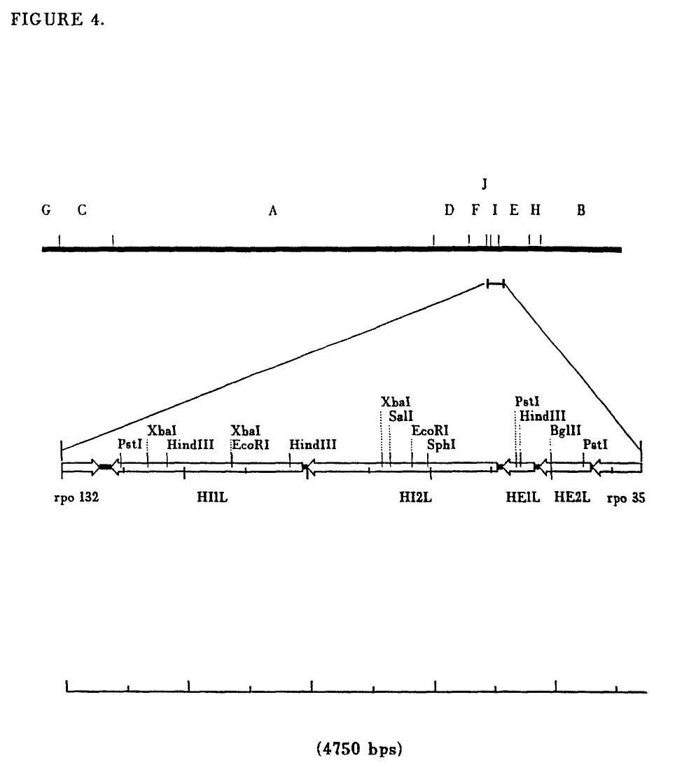

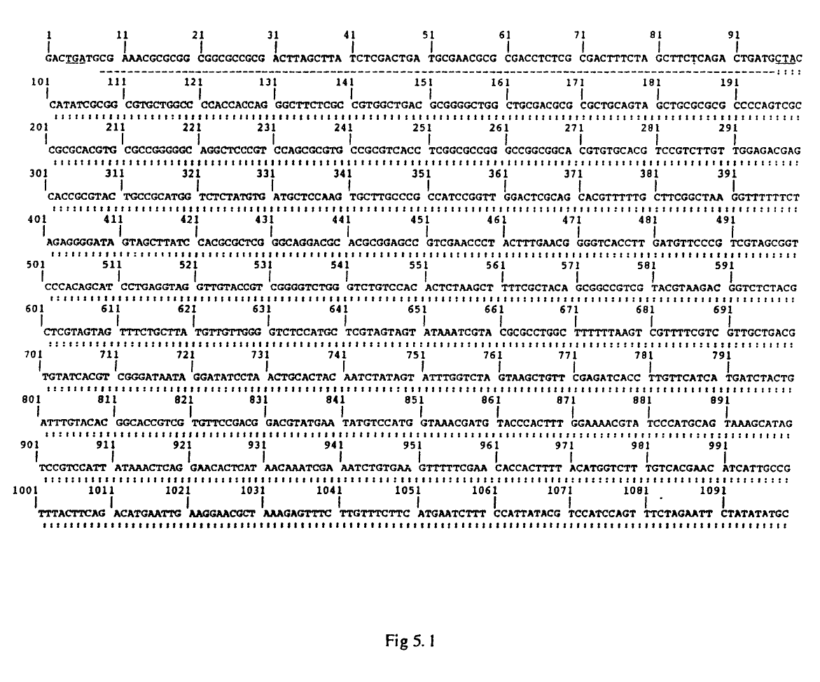

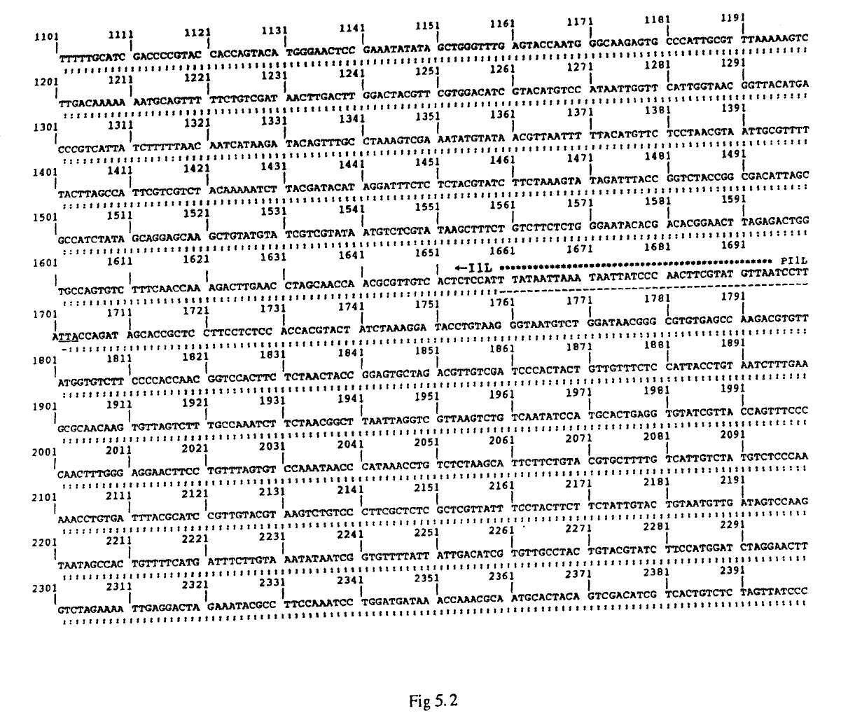

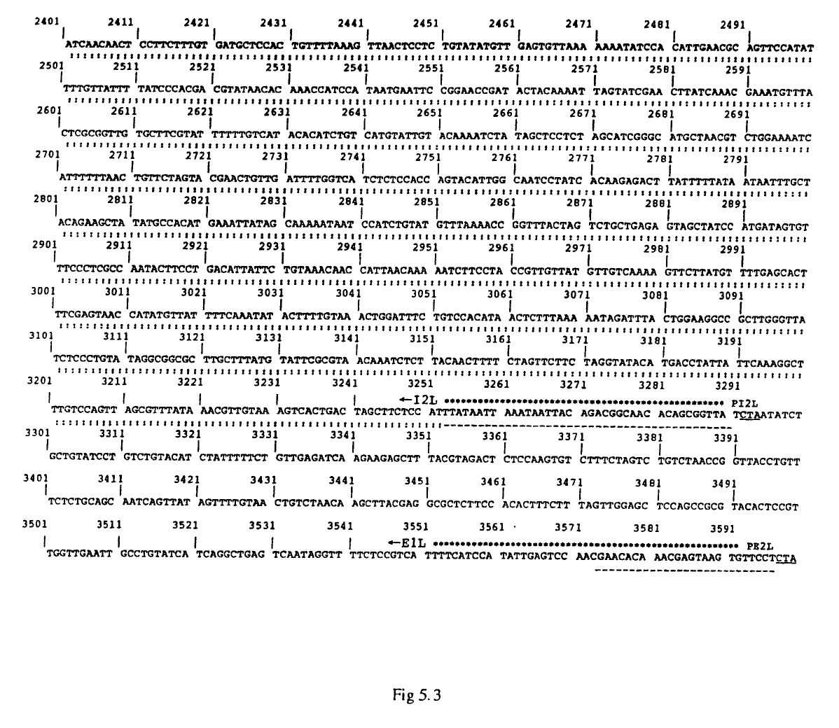

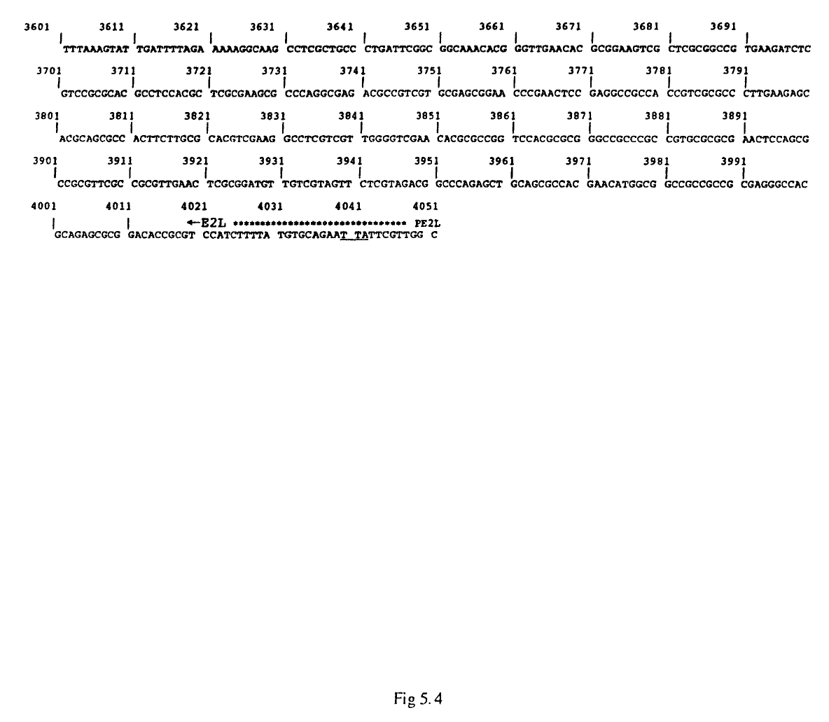

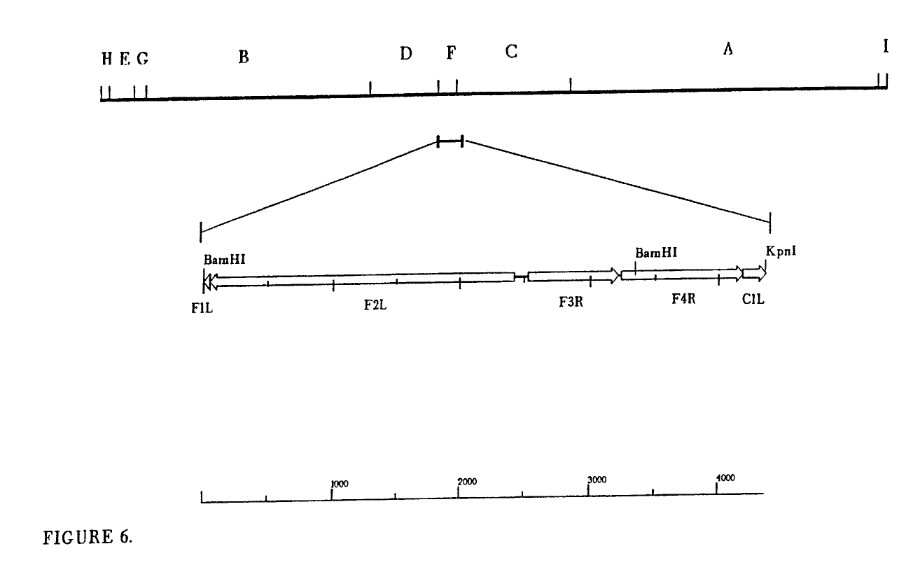

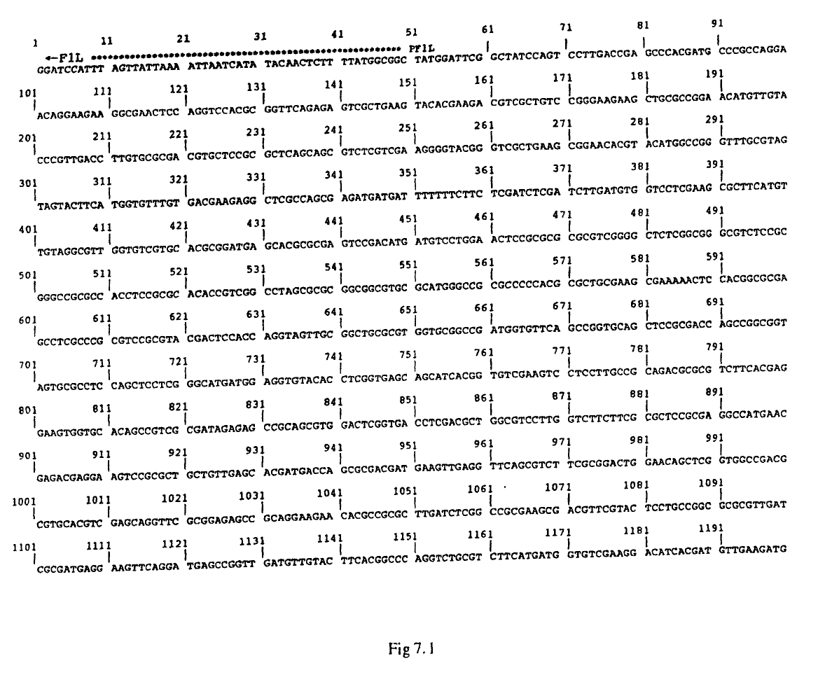

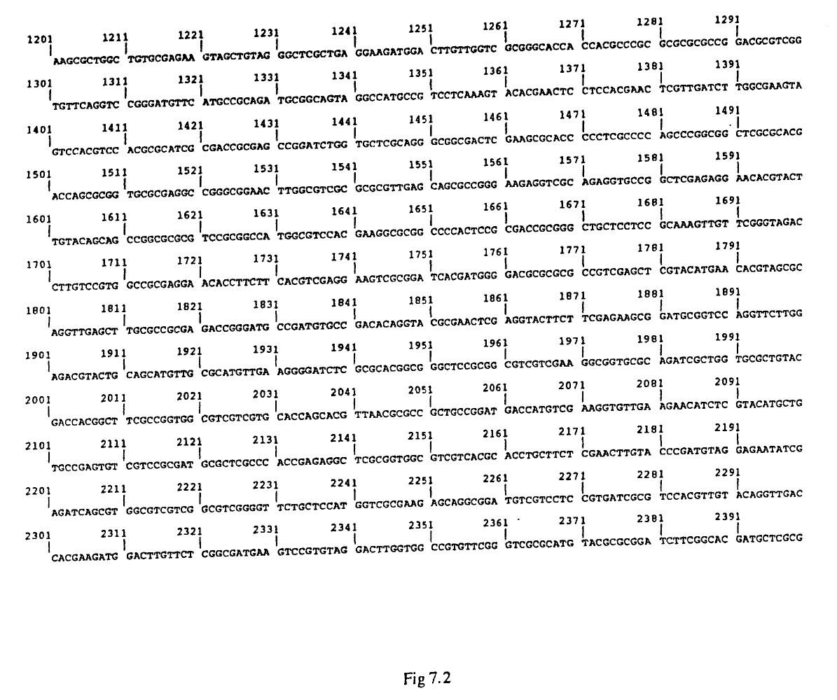

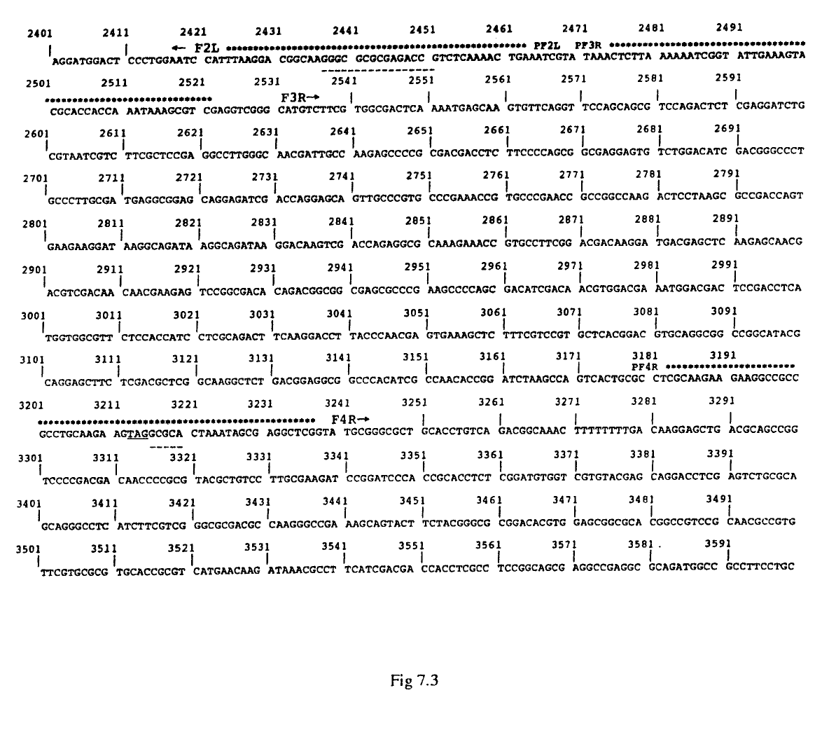

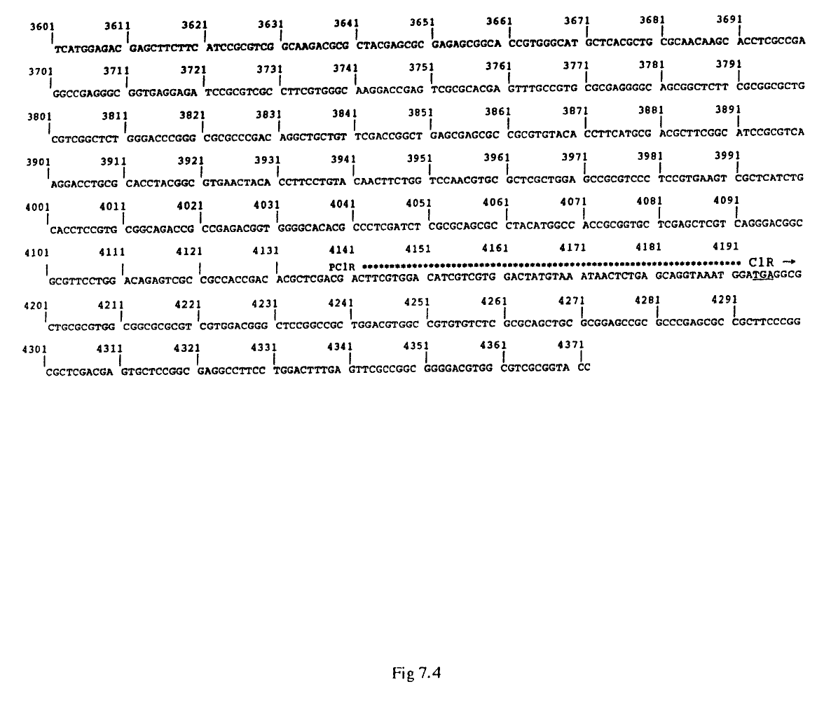

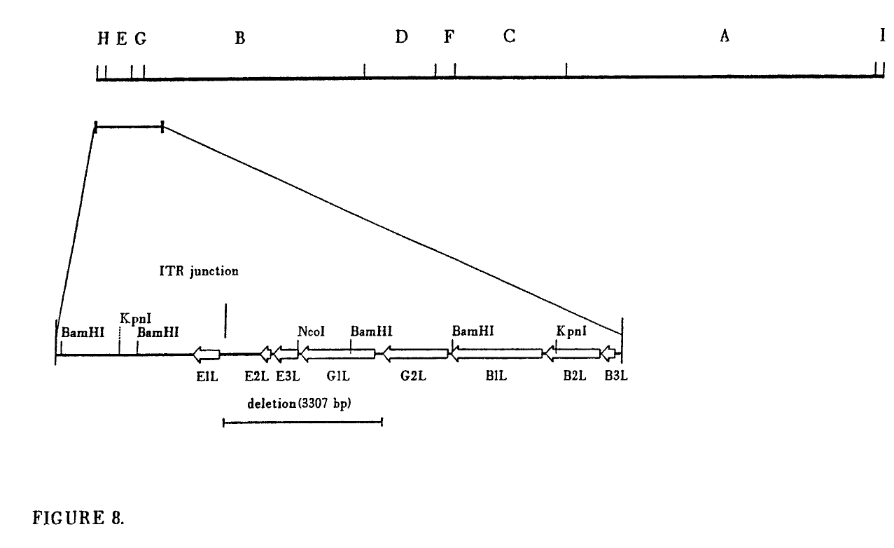

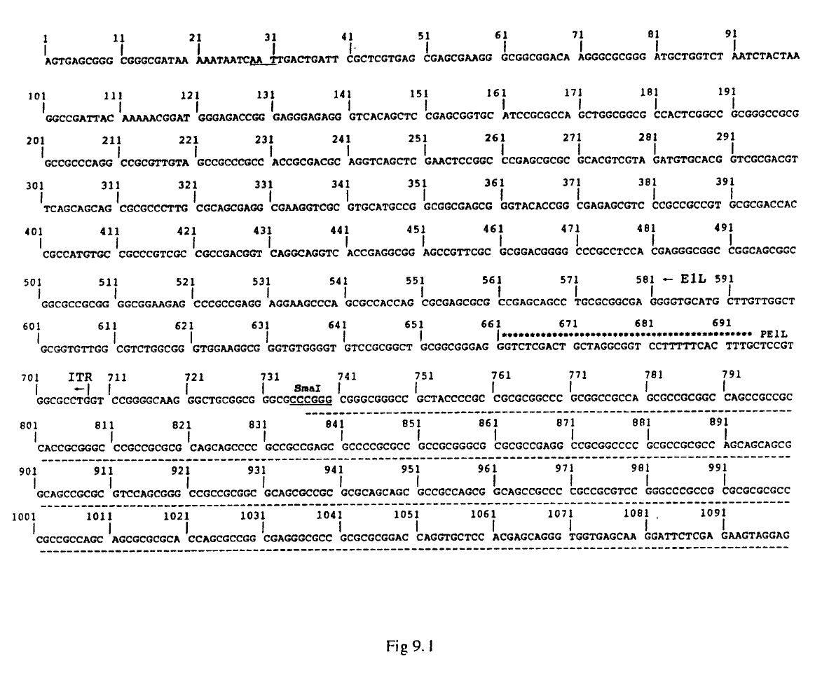

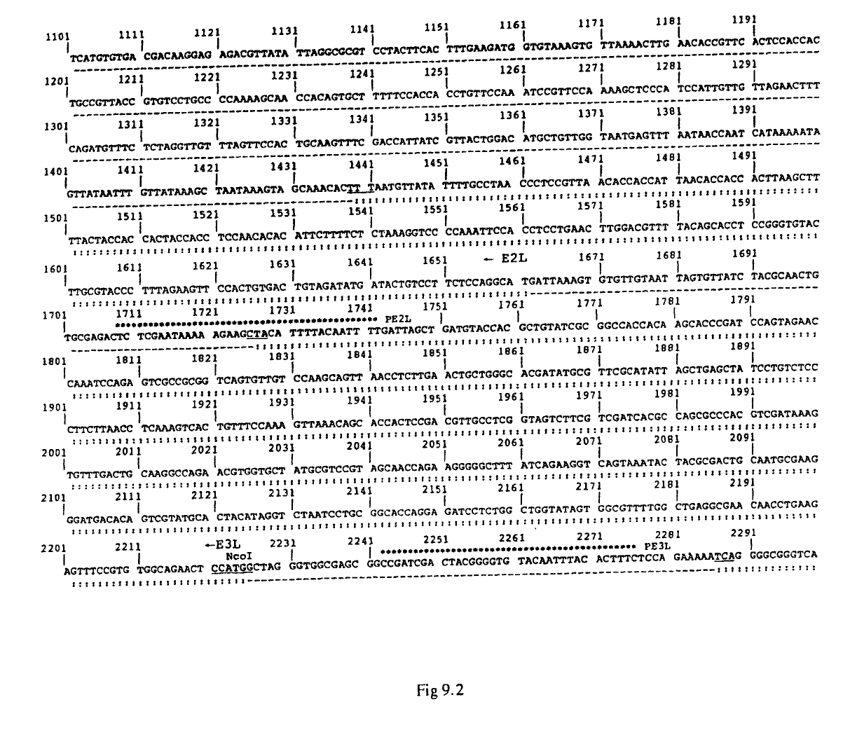

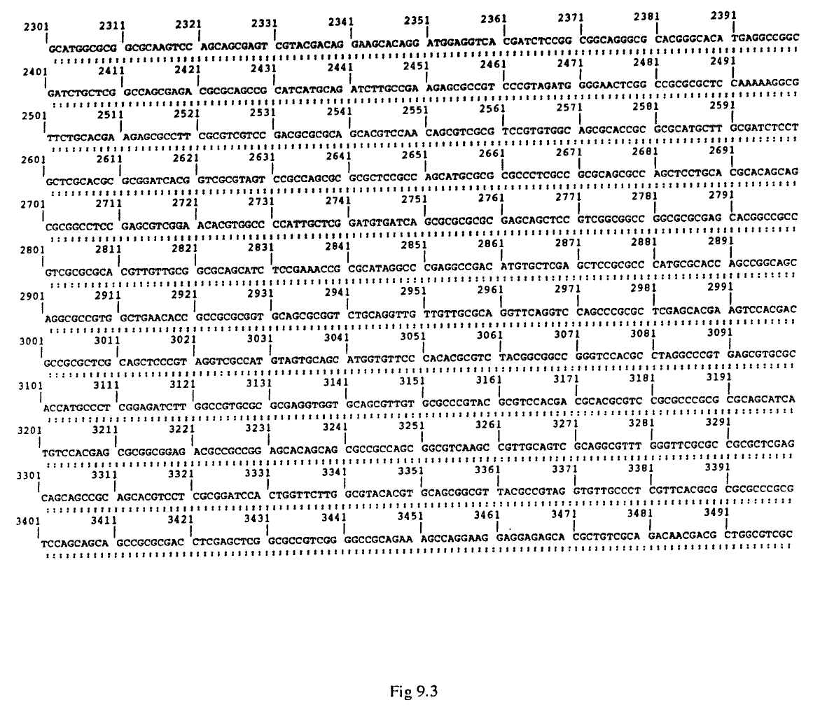

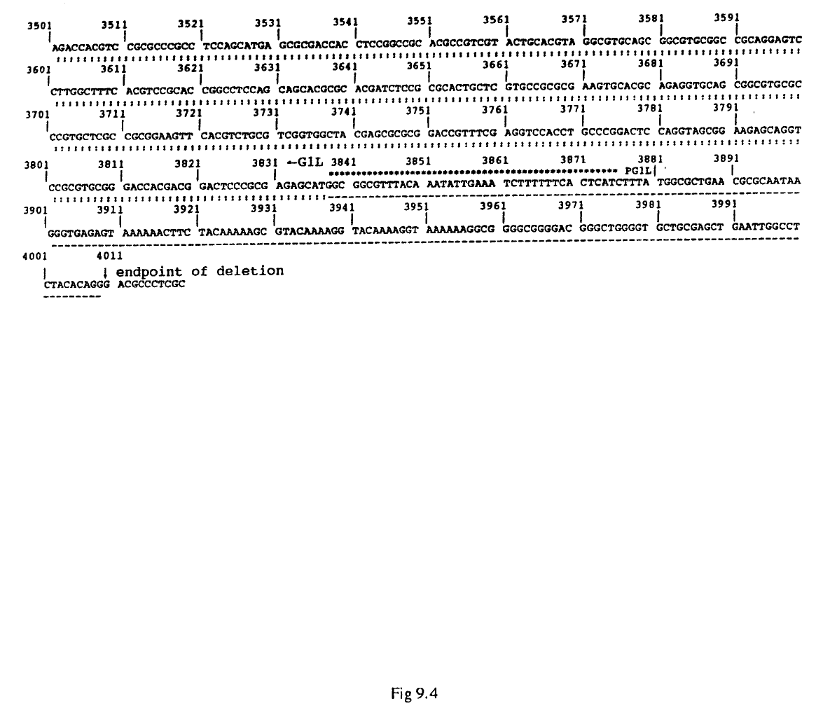

Potential insertion sites are contained within restriction fragments KpnI E of strain NZ-2, KpnI D of strain NZ-7 and KpnI D of strain NZ-10. Potential insertion sites are located in the restriction fragments BamHI E and BamHI G of strain NZ-2 shown in FIGS. 8 and 9. Other potential insertion sites have been identified as intergenic regions lying between regions encoding viral genes. Further examples are illustrated in FIGS. 4 and 5 (restriction fragments HindIII F, J, I and E of strain NZ-2) and in FIGS. 6 and 7 (restriction fragments BamHI F and C of strain NZ-2). Other insertion sites are also within the scope of the invention, for example, any non-essential gene or intergenic region within the orf virus genomic DNA sequence. Moreover, one or more insertion sites may be selected and used at a time.

There are two currently preferred insertion sites. The first of these sites is the intergenic region between RNA polymerase subunit gene, rpo 132 and the open reading frame of the presumptive gene (H) I1L (FIG. 4). As shown in FIG. 5 this insertion site is 90 nucleotides in length, extending from positions 11 to 96.

The second of the preferred insertion sites is the NcoI site located at the beginning of gene E3L (FIG. 8). As shown in FIG. 9 this insertion sited is 61 nucleotides in length, extending from positions 2226 to 2286.

As will also be appreciated, if expression of the foreign gene is to be achieved, it must be under the control of a transcriptional promoter capable of expressing that gene.

A description of poxvirus promoters can be found in Moss, B. (1990). Regulation of vaccinia virus transcription. Annu Rev Biochem. 59, 661-688 incorporated herein by reference. As has been shown, poxvirus RNA polymerase complexes responsible for copying the gene to make a mRNA, will transcribe any gene that is preceded by a poxvirus promoter.

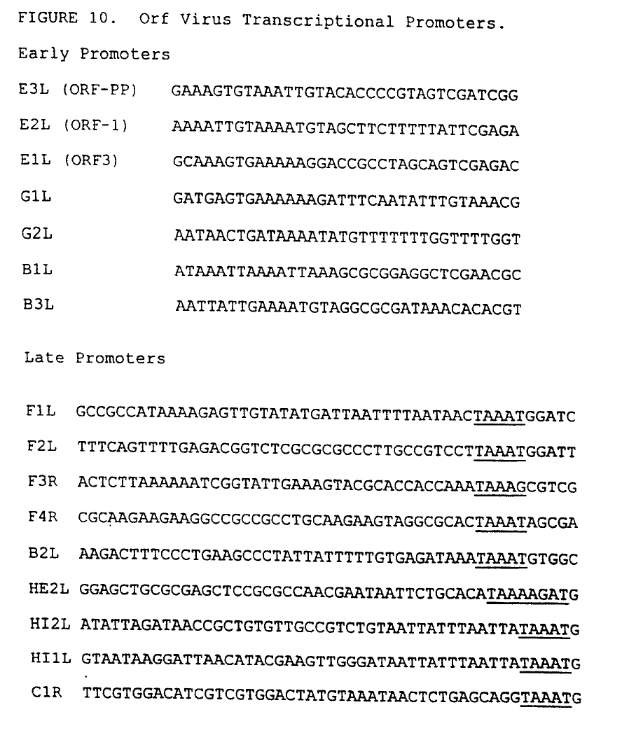

Preferably therefor, the promoter used will be a poxvirus promoter, and particularly a parapoxvirus promoter. The presently preferred promoter is an orf virus promoter. The orf virus promoter may be an early, intermediate or late promoter. Nucleotide sequencing has allowed the identification of a number of orf virus transcriptional promoters including early, intermediate and late promoters. Orf virus early and late promoters are shown in FIG. 10.

One preferred orf virus promoter is the early promoter of the putative gene E1L originally described as ORF-3 by Fraser, K. M., Hill, D. F., Mercer, A. A. and Robinson, A. J. (1990). Sequence analysis of the inverted terminal repetition in the genome of the parapoxvirus, orf virus. Virology. 176, 379-389 and Fleming, S. B., Fraser, K. M., Mercer, A. A. and Robinson, A. J. (1991). Vaccinia virus-like early transcriptional control sequences flank an early gene in the orf parapoxvirus. Gene. 97, 207-212.

Of the late promoters PF1L and PF3R are preferred. Initial studies on the relative strengths and the temporal expression of the promoters indicate that PF3R is an early-late promoter and is therefore the presently preferred promoter for expressing cloned genes encoding antigenic polypeptides. PF1L is a strong late promoter and is the presently preferred promoter for the expression of the β-galactosidase reporter gene. The orientation of the promoter and the gene it controls may be arranged as appropriate. Combinations of promoters may also be employed.

In a further aspect the invention consists in replicable transfer vectors suitable for use in preparing the modified orf virus vector of the invention. Replicable transfer vectors may be constructed according to techniques well known in the art (Sambrook, J, Fritsch, E. F. and Maniatis, T. Molecular Cloning, A Laboratory Manual (Second Edition) Cold Spring Harbour Laboratory Press 1989), or may be selected from cloning vectors available in the art.

The cloning vector may be selected according to the host cell to be used. Useful vectors will generally have the following characteristics:

(i) the ability to self-replicate;

(ii) the possession of a single target for any particular restriction endonuclease; and

(iii) desirably, carry genes for a readily selectable marker such as antibiotic resistance.

Two major types of vector possessing the aforementioned characteristics are plasmids and bacterial viruses (bacteriophages or phages). Plasmid vectors are preferred for use in the present invention. The plasmid vector will comprise a non-essential region of the orf virus genome, a foreign gene or genes under the control of one or more orf virus promoters, and a segment of bacterial plasmid DNA. The vector may be a linear DNA molecule but is preferably circular.

In the construction of a modified orf virus it is also an advantage to be able to distinguish the modified virus from the unmodified virus by a convenient and rapid assay. Such assays include measurable colour changes, antibiotic resistance and the like. For rapid assay purposes, the virus vector desirably further includes at least one reporter gene such as lacz, and and/or at least one selectable marker gene such as x-gpt.

In a preferred embodiment, the xanthine-guanine phosphoribosyltransferase gene (x-gpt) and the β-galactosidase gene are inserted into the plasmid vector under the control of suitable orf virus transcriptional promoters. The orientation of the inserted genes may also be important in determining whether recombinants can be recovered from transfections. FIG. 14 shows the x-gpt gene in different orientations in pVU-DL101 and pVU-DL102.

In a further aspect, the present invention provides a method for producing a modified orf virus vector. The method comprises transfecting the plasmid cloning vectors defined above into a selected host cell infected with orf virus. Suitable transfection techniques are well known in the art, for example, calcium phosphate-mediated transfection as described by Graham, F. L. and Van der Eb, A. J. (1973). A new technique for the assay of infectivity of human adenovirus type 5 DNA. Virology. 52, 456-467. Other techniques include electroporation, microinjection, or liposome or spheroplast mediated transfer but are not limited thereto. Preferably, liposome-mediated transfection is used. This method is described by Felgner, P. L., Gadek, T. R., Holm, M., Roman, R., Chan, H. W., Wenz, M., Northrop, J. P., Ringold, G. M. and Danielsen, M. (1987) Lipofection: a highly efficient, lipid-mediated DNA-transfection procedure. Proc Natl Acad Sci USA. 84, 7413-7417.

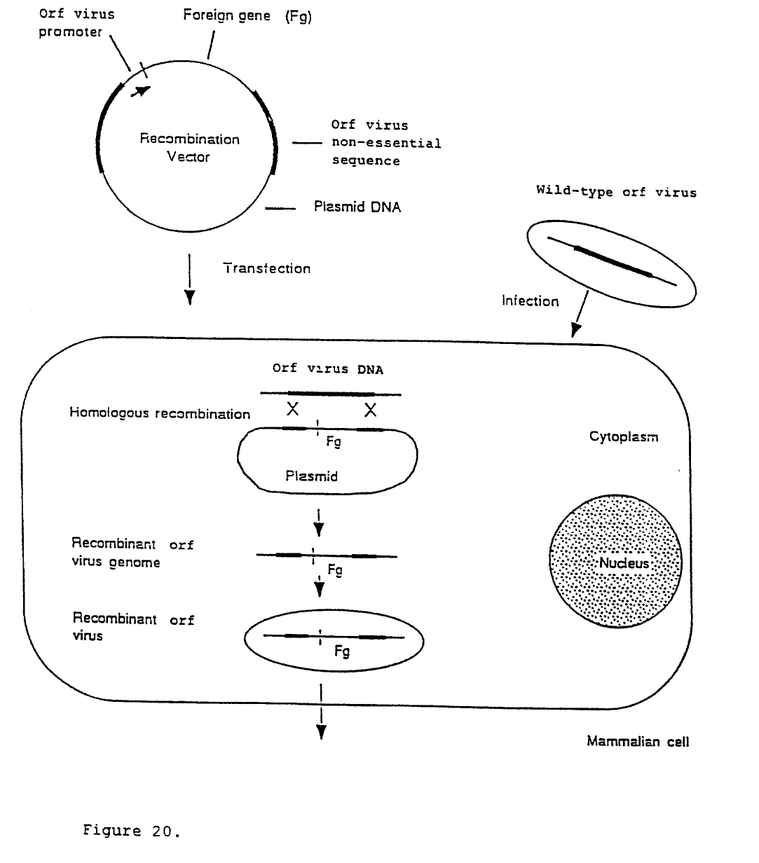

Upon transformation of the selected host with the cloning vector, recombinant or modified orf virus vectors may be produced. The modified virus may be detected by rapid assays as indicated above. For the preferred vectors the presence of the β-galactoside gene is detectable where clones give a blue phenotype on X-gal plates facilitating selection. Once selected, the vectors may be isolated from culture using routine procedures such as freeze-thaw extraction. Purification is effected as necessary using conventional techniques. A strategy for the generation of modified orf virus is shown in FIG. 20.

The transformed host cells also form part of the invention. Many host cells are known in the art including bacterial, insect, plant and animal cells. Preferably, the host cell is a eukaryotic cell. Mammalian host cells are particularly desirable. The preferred host cells of the present invention are primary bovine testis cells or primary ovine testis cells (lamb testis cells).

As will be appreciated, in a further aspect of the invention, the protocol described above may be used to prepare heterologous polypeptides as well as antigens.

In another aspect, the present invention comprises a vaccine preparation comprising the modified orf virus which contains exogenous antigenic DNA, or a fragment or variant thereof having equivalent immunological activity thereto in combination with a pharmaceutically acceptable diluent or carrier and optionally or alternatively an adjuvant. Examples of suitable adjuvants known to those skilled in the art include saponins, Freund's adjuvants, water-in-oil emulsions, glycerol, sorbitol, dextran and many others. Generally, adjuvants will only be used with non-living viral vaccine preparations.

In a further aspect, the present invention comprises a vaccine preparation comprising the modified orf virus which contains exogenous antigenic DNA in combination with exogenous DNA encoding cytokine genes or genes for other biological effector molecules which may modify or augment an existing immune response.

The vaccine may be formulated in any convenient physiologically acceptable form. Vaccine preparation techniques for smallpox are disclosed in Kaplan, Br. Med Bull. 25, 131-135 (1969).

Most usefully, the vaccine is formulated for parenteral administration. The term “parenteral” as used herein refers to intravenous, intramuscular, intradermal and subcutaneous injection.

In addition the vaccine may be formulated for oral administration.

Other therapeutic agents may also be used in combination with the vaccine.

Where necessary, the vaccine may be administered several times over a defined period to maximise the antibody response to the foreign antigen.

Other methods for inserting foreign genes into orf virus are also contemplated. Potentially, a restriction endonuclease that cuts orf virus DNA once may be used. The cleaved site may be removed following in vitro mutagenesis followed by joining by ligation. If the site is in an essential gene the mutagenesis may be arranged such that the gene function is not affected. This is possible by substituting a base in a codon that lies wholly or partly in the restriction endonuclease cleavage site with another base that allows the new codon to code for the same amino acid but for that substitution to remove the cleavage site for that particular restriction endonuclease. The cleavage site could then be created within any non-essential gene by mutagenesis. This cleavage site then acts as a site for the insertion of foreign genes. The insertion of foreign genes may be done outside the cell by removing the phosphate from the cleaved ends of the DNA to prevent recreation of uninterrupted orf virus DNA, joining a foreign gene which has phosphorylated ends into the orf virus DNA in a ligation reaction and then transfecting the resulting ligation mixture into cells permissive for orf virus. To recover the virus the cell is infected with a poxvirus that was non-permissive for those cells, for instance fowlpox virus and primary bovine testis cells.

Non-limiting examples will now be provided.

### EXAMPLE 1

### Selection of a Suitable Cell Culture System

The source of cells for culture in the methods described in this application was calves of between one day and three months of age. The testicles were removed from the scrotum of the animal without anaesthetic by a veterinarian skilled in this procedure. The testicles were removed with the tunica parietalis intact to keep the culture cells sterile. The tissue was transported on ice to the laboratory, and the testicular tissue removed from the testis, dispersed into single cells and small aggregates of cells and incubated in suitable culture vessels in culture medium by sterile procedures familiar to those skilled in the art.

### EXAMPLE 2

### Identification of Insertion Sites

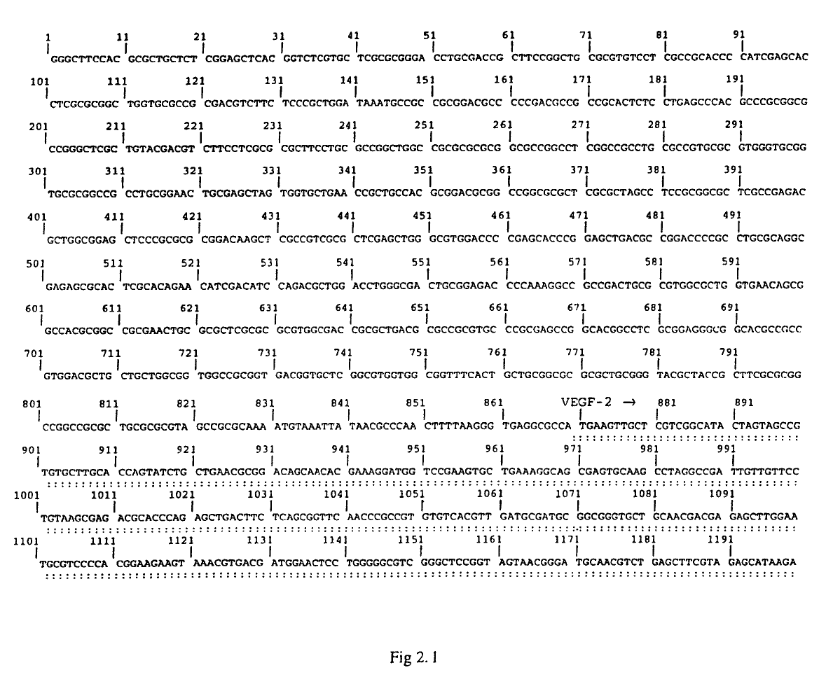

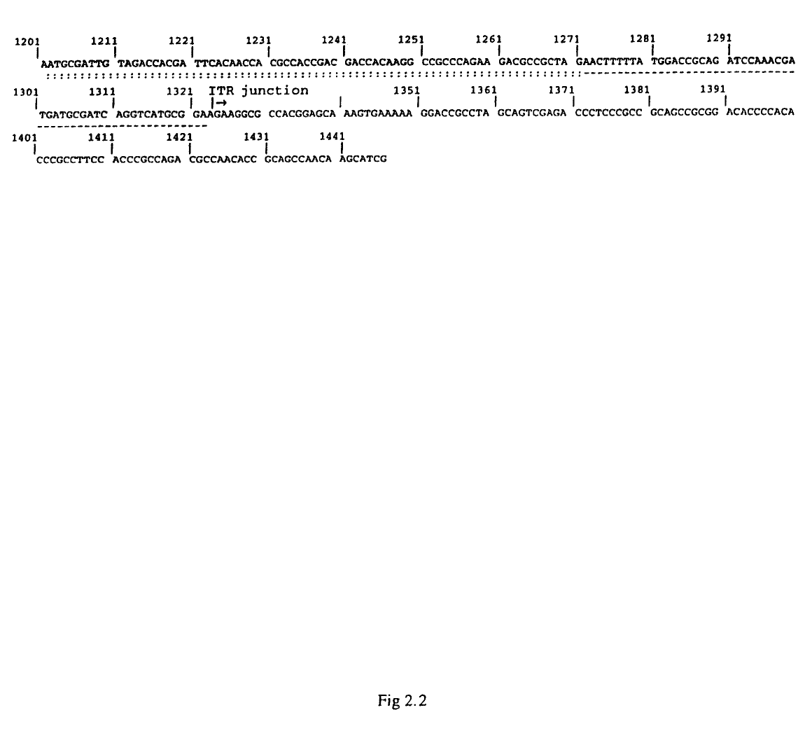

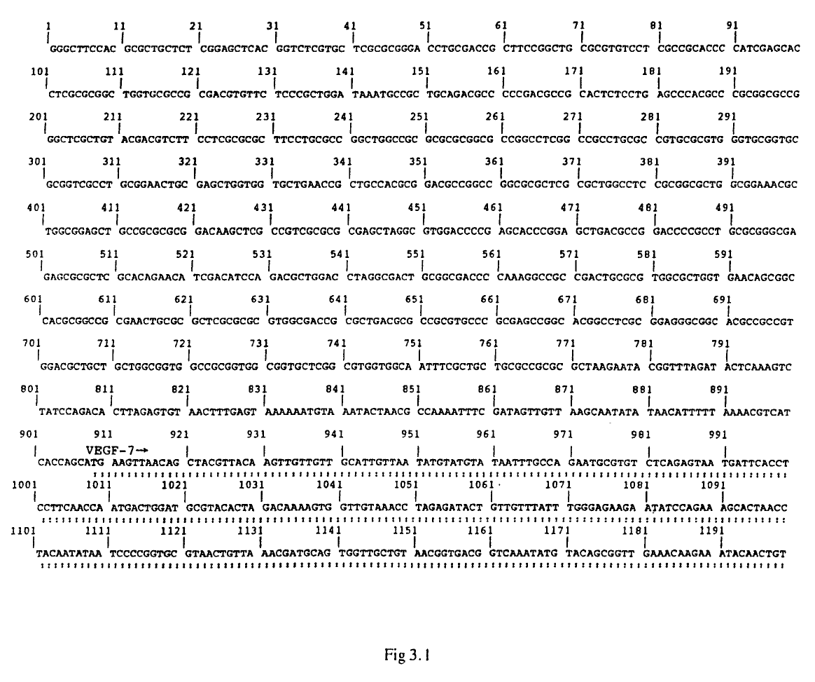

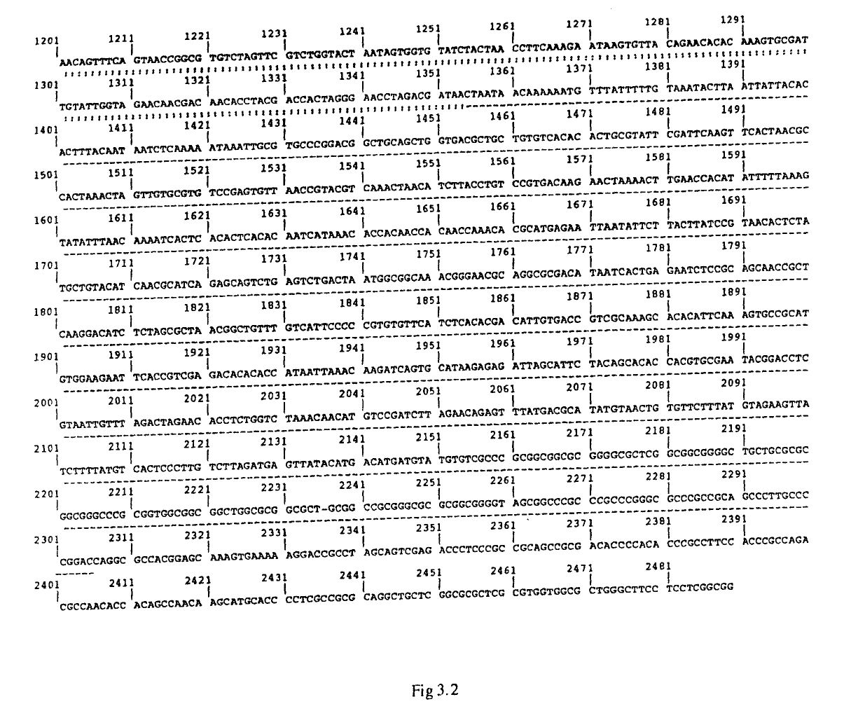

The DNAs of various orf virus isolates have been physically mapped using restriction endonucleases. Such mapping has revealed that there are many different strains of the virus that can be distinguished by the size and order of the restriction endonuclease-generated fragments although strains may not necessarily differ in their phenotype. From this data it was noted that there was a difference in size between two strains in a restriction endonuclease KpnI fragment mapping to the right end of the genome (Robinson A. J., Barns. G., Fraser, K. Carpenter, E. and Mercer, A. A. (1987). Conservation and variation in orf virus genomes. Virology. 157, 13-23). These two strains were designated NZ-2 and NZ-7 and the fragments KpnI E and KpnI D respectively. NZ-7 contained the larger of the two fragments. The difference in size was about 1 kilobase pair. Another strain designated NZ-10 was seen to have a fragment, fragment KpnI D intermediate in size between the corresponding fragments in NZ-2 and NZ-7 but located in the same relative position in the genome (see FIG. 1). This variability suggested that all or part of the region was non-essential and that within this fragment, a site in which to insert foreign DNA might be found. The regions described have subsequently been sequenced and potential insertion sites identified (FIG. 2 and FIG. 3).

Another potential insertion site was identified when DNA/DNA hybridization between strains, for example between NZ-2 and NZ-7, detected a region of non-homology extending over 2.75 kilobase pairs and this was mapped to a region about 30 kilobase pairs from the right end of the genome (Robinson A. J., Barns, G., Fraser, K, Carpenter, E. and Mercer, A. A. (1987). Conservation and variation in orf virus genomes. Virology. 157, 13-23 and Naase, M., Nicholson, B. H., Fraser, K. M., Mercer, A. A. and Robinson, A. J. (1991). An orf virus sequence showing homology to the fusion protein gene of vaccinia virus. J. Gen Virol. 72, 1177-1181) (FIG. 4). This region was then completely sequenced and two genes, HI1L and HI2L identified, each of which contains potential insertion sites (FIG. 5).

A third potential insertion site was located in the centre of the genome where a size difference of 100 base pairs was seen between the BamHI G fragment in a strain designated NZ-41 and equivalent region in the other strains examined (Robinson. A. J., Barns, G., Fraser, K., Carpenter, E. and Mercer, A. A. (1987). Conservation and variation in orf virus genomes. Virology. 157, 13-23). The nucleotide sequence of the equivalent region in the genome of strain NZ-2, the BamHI F fragment, has been determined and two potential insertion sites identified (FIG. 6 and FIG. 7).

Fourthly, a spontaneous re-arrangement of the orf virus genome of strain NZ-2 was detected following serial propagation of the virus in cell culture. This re-arrangement resulted in the addition of 16 kilobase pairs of right-end DNA sequences to the left end and the deletion of 3.3 kilobase pairs of DNA from the left end. Genomic analysis of a transposition-deletion variant of orf virus reveals a 3.3 kbp region of non-essential DNA (Fleming S. B., Lyttle, D. J., Sullivan, J. T., Mercer, A. A. and Robinson, A. J. (1995). J Gen Virol., 76, 2969-2978). The order of nucleotides making up the region of the genome that can tolerate a deletion has been deduced by the method of Sanger and three genes contained therein identified. These genes correspond to E2L, formerly ORF-1 (Fraser, K. M., Hill, D. F., Mercer, A. A. and Robinson, A. J. (1990). Sequence analysis of the inverted terminal repetition in the genome of the parapoxvirus, orf virus. Virology. 176, 379-389), E3L formerly ORF-PP (Mercer, A. A., Fraser, K., Stockwell, P. A. and Robinson, A. J. (1989). A homologue of retroviral pseudoproteases in the parapoxvirus, orf virus. Virology 172, 665-668) and G1L (Sullivan, J. T., Fraser, K., Fleming, S. B., Robinson, A. J. and Mercer, A. A. (1995). Sequence and transcriptional analysis of an orf virus gene encoding ankyrin-like repeat sequences. Virus Genes, 9, 277-282). This region (FIG. 8) is another potential site for gene insertion (see FIG. 9).

### EXAMPLE 3

### Identification of Orf Virus Promoters

Determining the nucleotide sequence of selected regions of the orf virus genome has allowed the identification of a number of orf virus transcriptional promoters, in the first instance by virtue of their similarity to other poxvirus transcriptional promoters, and later by functional assays.

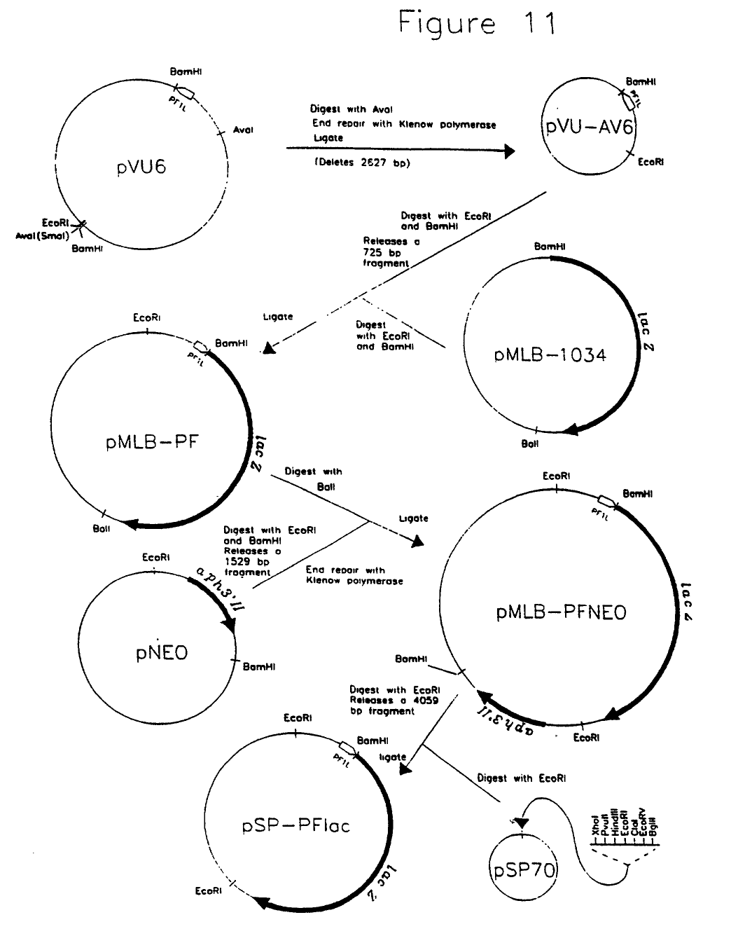

Orf virus early and late promoters are shown in FIG. 10. The early promoter E1L (ORF-3) was shown to make mRNA early in the cell cycle (Fleming, S. B., Fraser, K. M., Mercer, A. A. and Robinson, A. J. (1991). Vaccinia virus-like early transcriptional control sequences flank an early gene in the orf parapoxvirus. Gene. 97, 207-212) and the late promoter F1L was deduced to be a late promoter by virtue of its similarity to a vaccinia virus late promoter. The orf virus late promoter is functional in a transient assay. Such assays have been described for instance by (Cochran, M. A., Mackett, M. and Moss, B. (1985). Eukaryotic transient expression system dependent on transcription factors and regulatory DNA sequences of vaccinia virus. Proc Natl Acad Sci USA. 82, 19-23). A third promoter F3R, identified as an early-late promoter, is also shown to be functional in a transient assay. The construction of a plasmid pSP-PFlac containing the orf virus late promoter, F1L, and the E. coli gene for β-galactosidase (lacz) such that the β-galactosidase gene is under the control of the orf virus late promoter is described in Example 6 and illustrated in FIG. 11.

(A) Assessment of Promoter Activity in Transient Assay

To show that the promoter is active in a transient assay, a confluent monolayer of bovine testis cells, in a plastic flask of 25 cm2 surface area for the adherence of the cells and suitable for cell culture work, was infected with orf virus at a multiplicity of infection of approximately 10 plaque forming units per cell. Two hours after infection, the plasmid containing the lacz gene linked to the promoter under investigation was introduced into orf virus infected bovine testis cells using the liposome mediated transfer technique as described by (Felgner, P. L., Gadek, T. R., Holm, M., Roman, R., Chan, H. W., Wenz, M., Northrop, J. P., Ringold, G. M. and Danielsen, M. (1987). Lipofection: a highly efficient, lipid-mediated DNA-transfection procedure. Proc Natl Acad Sci USA. 84, 7413-7417) and as set forth in Example B. Forty eight hours after infection, 35 μl of a solution of 5-bromo-4-chloro-3-indolyl-β-D galactosidase (X-gal) at a concentration of 2% w/v in water was added to 1 ml of 1% agarose in cell culture medium which was overlayed onto the cells after the removal of the liquid medium and allowed to form a gel at room temperature (in the range of 15°-25° C.). Over the succeeding 24 hours the development of a blue coloration in the cells and in the gel above the affected cells was looked for. The development of a blue coloration greater than that seen in cells treated similarly, but with a plasmid containing the β-galactosidase gene not under control of a transcriptional promoter, indicated that the promoter being tested was active.

In a further aspect of investigating promoter function a quantitative assay for β-galactosidase activity in transiently-infected bovine testis cells is performed. Cells are grown as confluent monolayers in multiwell plastic tissue culture trays containing 24 wells 1.5 cm in diameter. Individual wells are infected with orf virus at a moi of 10 and two hours after infection the plasmid construct containing the promoter linked to the β-galactosidase gene is introduced into the infected cells using the liposome mediated transfection technique described above. Cells are harvested by scraping into a 1 ml volume of phosphate-buffered saline (PBS), collected by centrifugation, washed with PBS and resuspended in a 200 μl volume of PBS. Cells are disrupted by three cycles of freezing and thawing, centrifuged, and the supernatant retained for the enzyme assay. The assay for β-galactosidase is conveniently performed in 96-well microtitre trays. The reaction mire of 0.1 ml contains 100 mM Na-phosphate, pH 7.3, 1 mM MgCl2, 50 mM β-mercaptoethanol, O-nitrophenyl-β-D-galactoside (ONPG) at a final concentration of 1.3 mg/ml and a 10-20 μl aliquot of the cell lysate. The reaction mix is incubated at 37° C. for 1 hour and the reaction is terminated by the addition equal volume of 1M NaCO3. The absorbance of each well is measured at 420 nm using a microtitre plate reader. The absorbance value is proportional to the amount of β-galactosidase activity present in the original extract and this enables the time course of expression and the relative strength of each promoter construct to be determined.

### EXAMPLE 4

### Construction of a Vector Plasmid Suitable for the Insertion of Foreign Genes into the Orf Virus Genome

The choice of non-essential DNA was the region discovered to be deleted in a re-arranged mutant of orf virus and the relevant sequence of nucleotides in this region can be found in Fraser, K. M., Hill, D. F., Mercer, A. A. and Robinson, A. J. (1990). Sequence analysis of the inverted terminal repetition in the genome of the parapoxvirus, orf virus. Virology. 176, 379-389 and in Sullivan, J. T., Fraser, K. M., Fleming, S. B., Robinson, A. J. and Mercer, A. A. (1995). Sequence and transcriptional analysis of an orf virus gene encoding ankyrin-like repeat sequences. Virus Genes 9, 277-282 and is shown in FIG. 8. The orf virus promoters used were an early promoter, E1L, described in Fraser, K. M., Hill, D. F., Mercer, A. A. and Robinson, A. J. (1990). Sequence analysis of the inverted terminal repetition in the genome of the parapoxvirus, orf virus. Virology. 176, 379-389 and Fleming, S. B., Fraser, K. M., Mercer, A. A. and Robinson, A. J. (1991). Vaccinia virus-like early transcriptional control sequences flank an early gene in the orf virus. Gene. 97, 207-212 and a late promoter F1L (Fleming, S. B., Blok, J., Fraser, K. M., Mercer, A. A. and Robinson, A. A. (1993). Conservation of gene structure and arrangement between vaccinia virus and orf virus. Virology. 195, 175-184) as shown in FIG. 10. The foreign genes chosen to demonstrate the process of creating a mutated orf virus were the E. coli β-galactosidase gene, which has the advantage that when expressed the protein product can be detected by a colour reaction (Miller, J. H. (1972). “Experiments in Molecular Genetics.” Cold Spring Harbor Laboratory, Cold Spring Harbor, N.Y. Moss, B. (1990). “Poxviridae and their Replication” in Virology, Fields et al., eds, 2nd ed. Raven Press, New York, 2079-2111), and the E. coli guanyl phoshoribosyl transferase (x-gpt) gene which when expressed can be used to select mutants from unmutated virus (Mulligan, R. C. and Berg, P. (1980). Expression of a bacterial gene in mammalian cells. Science. 209, 1422-1427). The following is a description of the construction of the vector plasmid. FIGS. 11-13 outline the construction in diagrammatic form.

(A) Cloning an Orf Virus Late Promoter in Front of the E. coli LacZ Gene

In the construction of a mutant orf virus it is an advantage to be able to distinguish mutant virus from unmutated virus by a convenient and rapid assay. Such an assay is provided by inserting the E. coli gene for the β-galactosidase enzyme under control of an orf virus transcriptional promoter into the vector plasmid. The late orf virus promoter was identified by determining the nucleotide sequence of a fragment of orf virus DNA designated BamHI F (Fleming, S. B., Blok, J., Fraser, K. M., Mercer, A. A. and Robinson, A. A. (1993). Conservation of gene structure and arrangement between vaccinia virus and orf virus. Virology. 195, 175-184). The sequence of the promoter F1L used in this construction is shown in FIG. 10. A sufficient quantity of the late promoter for the construction can be obtained from the plasmid designated pVU-6 which has been described (Mercer, A. A., Fraser, K., Barns, G. and Robinson, A. J. (1987). The structure and cloning of orf virus DNA. Virology. 157, 1-12). A total of 2.62 kb of DNA is deleted from the BamHI F fragment of orf NZ-2 by digesting the plasmid pVU-6, which contains the BamHI F fragment of orf NZ-2 cloned into the plasmid pUC-8 (Viera, J. and Messing, J. (1982). The pUC plasmids, an M13mp7 derived system for insertion mutagenesis and sequencing with synthetic universal primers. Gene. 19, 259-268) with AvaI. This enzyme cleaves the SmaI site of the pUC-8 polylinker and six internal AvaI sites in BamHI E. The AvaI sites remaining on the vector fragment are end-filled with Klenow DNA polymerase, and religated to give the plasmid pVU-Av6. The plasmid pVU-Av6 is cut with BamHI and EcoRI releasing a 725 bp fragment containing the orf virus late promoter. This fragment is cloned into pMLB 1034 (Weinstock, G. M., Berman, M. L. and Silhavy, T. J. (1983). “Chimeric genetics with β-galactosidase in gene amplification and analysis,” in Expression of Cloned Genes in Procaryotic and Eucaryotic Cells, Papas et al., eds. Elsevier, New York, 27-64) which contains a “headless” lacz gene. This cloning places the orf virus late promoter in front of lacz and supplies it with an ATG initiation codon allowing the synthesis of β-galactosidase. The colonies that result from this cloning step give a blue phenotype on X-gal plates facilitating the selection of the required clone. A unique BalI site downstream from the lacz insert of pMLB-1034 is converted to an EcoRI site by the following cloning steps. The Tn5 aminoglycoside 3′ phosphotransferase gene is released from the plasmid pNEO (Beck, E., Ludwig, A., Aurswald, E. A., Reiss, B. and Schaller, H. (1982). Nucleotide sequence and exact location of the neomycin phosphotransferase from transposon Tn5. Gene. 19, 327-336) with EcoRI and BamHI. The restriction sites are end-filled with Klenow DNA polymerase and the fragment ligated into plasmid pMLB-PF which had been cut with BalI. Recombinants are selected by plating on kanamycin medium. This creates an EcoRI or BamHI site at the position of the original BalI site depending on the orientation of the cloned aminoglycoside 3′-phosphotransferase II (aphII) gene. BalI often cuts DNA inefficiently, but the method allows for the selection of the plasmids which have been cut by BalI and have received the insert, consequently becoming modified in the desired manner. The plasmid pMLB-PFneo is cut with EcoRI and a 4059 bp EcoRI fragment containing the PF-lacZ fusion is cloned into pSP-70 (Melton, D. A., P. A., R., Rebagliati, M. R., Maniatis. T., Zinn, R. and Green, M. R. (1984). Efficient in vitro synthesis of biologically active RNA and RNA hybridization probes from plasmids containing bacteriophage SP6 promoter. Nucleic Acids Res. 12, 7035-7056) at the EcoRI site to give the plasmid designated pSP-PFlac shown in the diagram FIG. 11.

(B) Cloning of an Orf Virus Early Promoter in Front of the E. coli X-GPT Gene

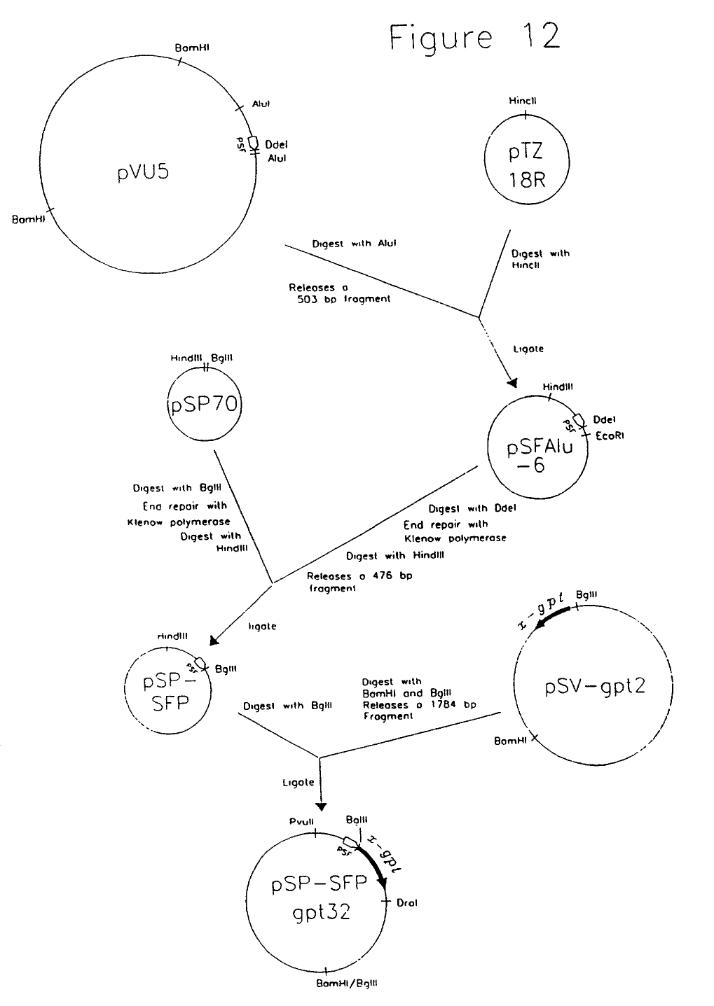

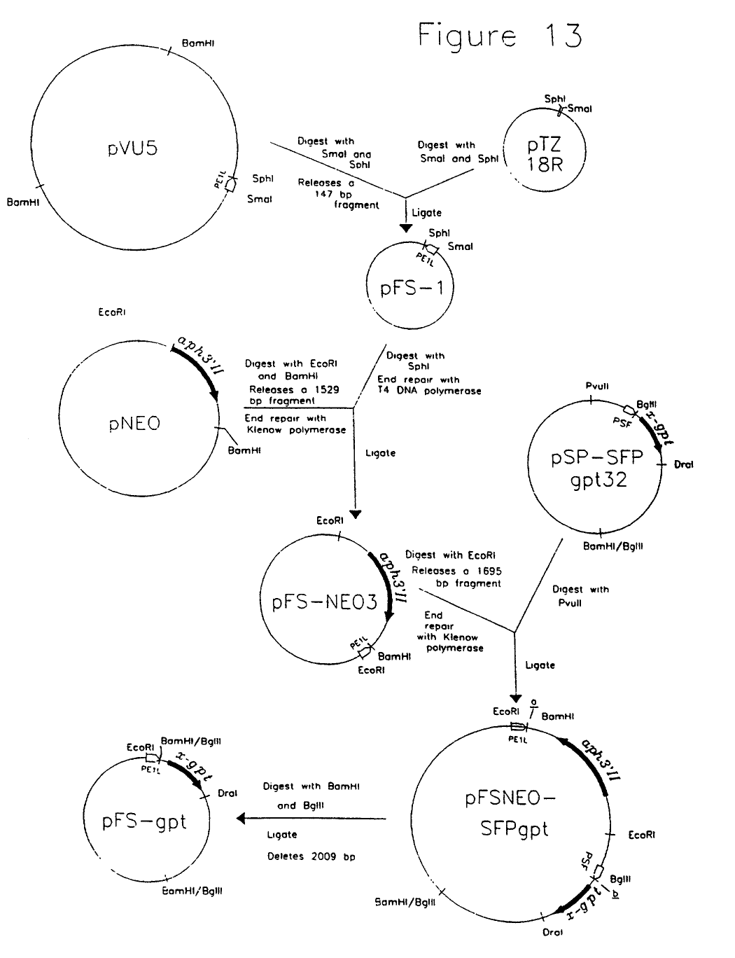

In the construction of the mutated orf virus, a means of selecting mutants from non-mutants, from a mixture of both, is required. A method that has been used by others is to utilise the guanyl phosphoribosyl transferase gene of E. coli. Resistance is conferred to a metabolic inhibitor, mycophenolic acid, when the gene is expressed in a eukaryotic cell. A method for incorporating this gene into a vector plasmid under the control of an early promoter is described by Falkner, F. G. and Moss, B. (1988). Escherichia coli gpt gene provides dominant selection for vaccinia virus open reading frame expression vectors. J Virol. 62, 1849-1854 and Boyle, D. B. and Coupar, B. E. (1988). Construction of recombinant fowlpox viruses as vectors for poultry vaccines. Virus Res. 10, 343-356. A plasmid designated pVU-5 is used to provide an early orf virus promoter. The plasmid pVU-5 contains the orf virus NZ-2 BamHI E fragment cloned into pUC-8 and the construction of this plasmid is described in Mercer, A. A., Fraser, K., Barns, G. and Robinson, A. J. (1987). The structure and cloning of orf virus DNA. Virology. 157, 1-12. An early promoter E1L has been described for the putative gene originally designated ORF-3 in pVU-5 by Fraser, K. M., Hill, D. F., Mercer, A. A. and Robinson, A. J. (1990). Sequence analysis of the inverted terminal repetition in the genome of the parapoxvirus, orf virus. Virology. 176, 379-389 and by Fleming, S. B., Fraser, K. M., Mercer, A. A. and Robinson, A. J. (1991). Vaccinia virus-like early transcriptional control sequences flank an early gene in the orf parapoxvirus. Gene. 97, 207-212; and it is this early promoter that is used in the method described in this application to construct a mutant orf virus. A 503 bp AluI A+T-rich fragment shown in the FIG. 12 is cleaved from pVU-5 and cloned into the HincII site of the multifunctional plasmid vector pTZ18R described in Mead, D. A., Szczesna-Skorupa, E. and Kemper, B. (1986). Single-stranded DNA “blue” T7 promoter plasmids: a versatile tandem promoter system for cloning and protein engineering. Protein Eng. 1, 67-74 giving pSFAlu-6. Plasmid pSFAlu-6 is cut with DdeI and the fragments end-filled with Klenow DNA polymerase. The fragments are recut with HindIII and a 467 bp HindIII-DdeI fragment ligated into pSP-70 which is prepared by cutting with BglII, end-filling and recutting with HindIII. The resulting plasmid pSP-SFP retains the BglII site which is reformed during the cloning step. The plasmid pSV-gpt2, containing the E. coli x-gpt gene, (Mulligan, R. C. and Berg, P. (1981). Selection for animal cells that express the Escherichia coli gene coding for xanthine-guanine phosphoribosyl transferase. Proc Natl Acad Sci USA. 78, 2072-2076) is cut with BamHI and BglII. This releases the x-gpt gene as a 1788 bp fragment which is then cloned into the BglII site of pSP-SFP, fusing the orf virus fragment to the x-gpt gene giving pSP-SFPgpt32. The plasmid pVU-5 is then cut with SmaI and SphI. A 150 bp SmaI-SphI fragment containing the early promoter E1L, the sequence of which is shown in FIG. 10, is cloned into pTZ18R between the SmaI and SphI sites giving the plasmid pFS-1. The plasmid pFS-1 is cut with SphI and incubated with T4 DNA polymerase. The aphII gene is released from the plasmid pNEO with EcoRI and BamHI. The EcoRI and BamHI sites are end-filled with Klenow DNA polymerase and the fragment ligated into pFS-1. The resulting plasmid pFS-neo3 contains the aphII gene flanked by an EcoRI site and a BamHI site which lies between it and the early orf virus promoter. A result of these manipulations is that the SphI site distal to the early promoter is converted to a BamHI site. The aphII gene and the early promoter lie in a “head-to-head” orientation and may be removed by digestion with EcoRI. Next, the plasmid pSP-sSFPgpt32 is cut with PvuII. The aphII-early promoter construct was cut out of pFSneo3 with EcoRI, end-filled with Klenow DNA polymerase, and ligated into the PvuII site. A plasmid termed FSneo-SFPgpt which contains the early promoter running in the same direction as the 503 bp AluI fragment is selected. The plasmid FSneo-SFPgpt is cut with BamHI and BglII. This step removes the sequence between nucleotides a and b (FIG. 13) together with the aphII gene as a BamHI-BglII fragment. The vector fragment is subjected to electrophoresis in an agarose gel and then purified using the powdered glass milk method described by (Vogelstein, B. and Gillespie, D. (1979). Preparation and analytical purification of DNA from agarose. Proc Natl Acad Sci USA. 76, 615-619) and the free BamI and BglII termini ligated together fusing the early promoter to the x-gpt gene. The net result of the manipulations described in steps 4, 5, 6, and 7 (FIG. 13) was to replace the sequence between nucleotides a and b in pSP-SFPgpt32 with the FS promoter forming pFS-gpt.

### EXAMPLE 5

### Identification of a Non-Essential Region of the Orf Virus Genome and Insertion of this Site into a Plasmid

A gene coding, potentially, for a peptide of 159 amino acids was found from the sequencing of the 4.47 kb BamHI E fragment which spans the ITR junction of the orf virus genome. This was termed F3L (ORF-PP) and shows homology to an open reading frame in retroviruses (Mercer, A. A., Fraser, K. M., Stockwell, P. A. and Robinson, A. J. (1989). A homologue of retroviral pseudoproteases in the parapoxvirus, orf virus. Virology. 172, 665-668) and to E. coli dUTPase (McGeoch, D. J. (1990). Protein sequence comparisons show that the ‘pseudoproteases’ encoded by poxviruses and certain retroviruses belong to the deoxyuridine triphosphatase family. Nucleic Acids Res. 18, 4105-4110). A spontaneous mutant of orf virus isolated in the laboratory was found not to contain the E3L gene due to a complex rearrangement involving the deletion of part of the BamHI E fragment and duplication of DNA segments from the opposite end of the genome at that locus. The E3L gene is therefore non-essential and was chosen as a target for the insertion of foreign DNA and to demonstrate that orf virus could tolerate the insertion of a foreign gene. A 2587 bp SmaI-BamHI fragment (FIG. 14) containing the unique region of NZ-2 BamHI E is cut out of pVU-5 and cloned into pSP-70 cut with PvuII and BglII. The resulting plasmid, pVU-DL100 contains a unique NcoI site that lies between the coding sequence of the E3L gene and its promoter.

### EXAMPLE 6

### Insertion of the E. coli X-GPT and Lac Z Gene Constructs into pVU-DL100 to Create a Vector Plasmid

Plasmid pVU-DL100 is cut with NcoI and end-filled with Klenow polymerase. The E3L-gpt construct is cut from pFSP-gpt with EcoRI and DraI, end-filled with Klenow polymerase and ligated into pVU-DL100 at the NcoI site. Ligation of the end-filled EcoRI site of the insert to the end-filled NcoI site on the plasmid creates an EcoRI site upstream of the early promoter. The insert is recovered in two orientations, pVU-DL101 with the x-gpt gene running in the opposite direction to the pseudoprotease gene and pVU-DL102 with the x-gpt gene running in the same direction as the pseudoprotease gene. The F1L-lac construct is cut out of pSP-PFlac with EcoRI and cloned into the EcoRI sites of both pVU-DL101 and pVU-DL102. Four plasmids with different orientations of the inserted fragments are recovered from the cloning but only two, pVU-DL104 derived from pVU-DL101, and pVU-DL106 derived from pVU-DL102 which contain the E3L-gpt and F1L-lac in the “back-to-back” orientation are used for transfection experiments.

### EXAMPLE 7

### Constructing a Chimeric Gene Expressing the T ovis 45W Antigen

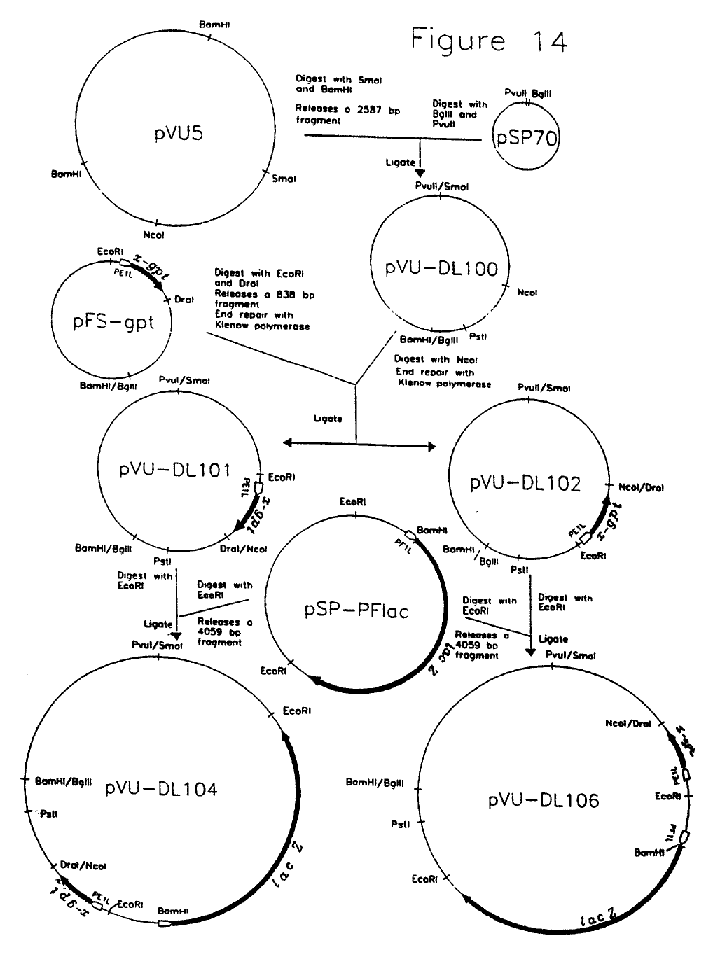

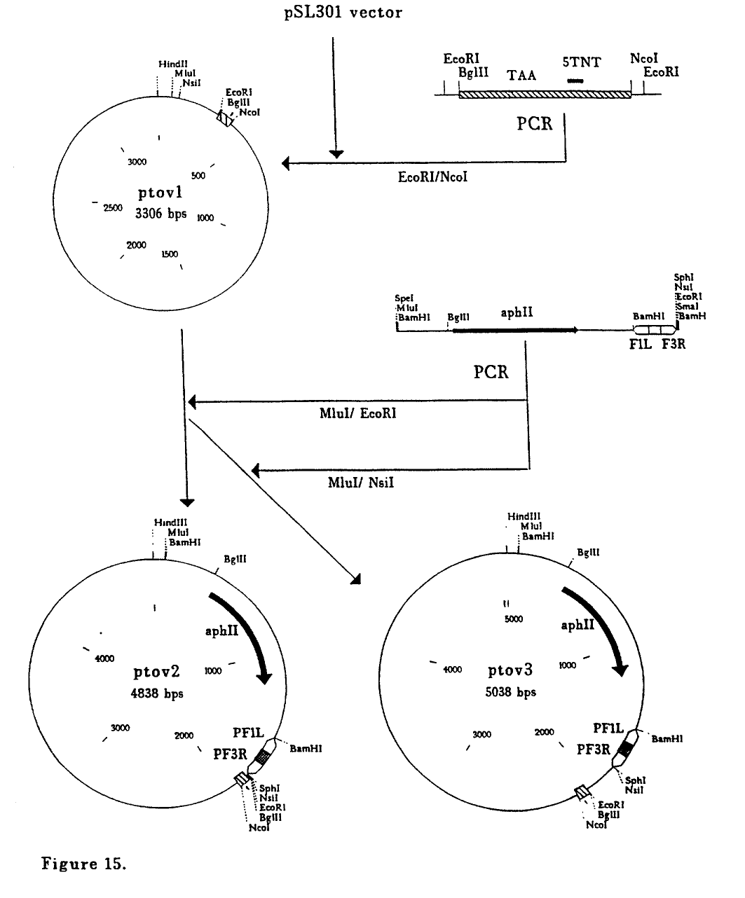

A 64 bp fragment of the VEGF like-gene from orf virus NZ-7 (Lyttle, D. J., Fraser, K. M., Fleming, S. B., Mercer, A. A. and Robinson, A. J. (1993) Homologs of vascular endothelial growth factor are encoded by the poxvirus orf virus. J Virol. 68, 84-92) containing five 3′ prime terminal codons, the translational termination codon TAA, and a poxvirus transcriptional terminator sequence 5TNT, was amplified using a pair of oligonucleotide primers designed to provide a BglII and a NcoI restriction site flanking the amplified sequence. This fragment was digested with BglII and NcoI and ligated into the vector pSL301 (Brosius, J. (1989) Superlinkers in cloning and expression vectors. DNA 8, 759-777) cut with BglII and NcoI to form the plasmid ptov1. A DNA fragment containing the aphII gene and the F1L and F3R promoters of orf virus was amplified by PCR using specific primers which introduced a MluI site at one end and a NsiI and EcoRI site at the other end. One portion of the amplified product was digested with MluI and EcoRI and ligated into ptov1 cut with MluI and EcoRI to create the plasmid ptov2. A second portion was digested with MluI and NsiI and ligated into ptov1 to form the plasmid ptov3. The steps showing this construction are illustrated in FIG. 15.

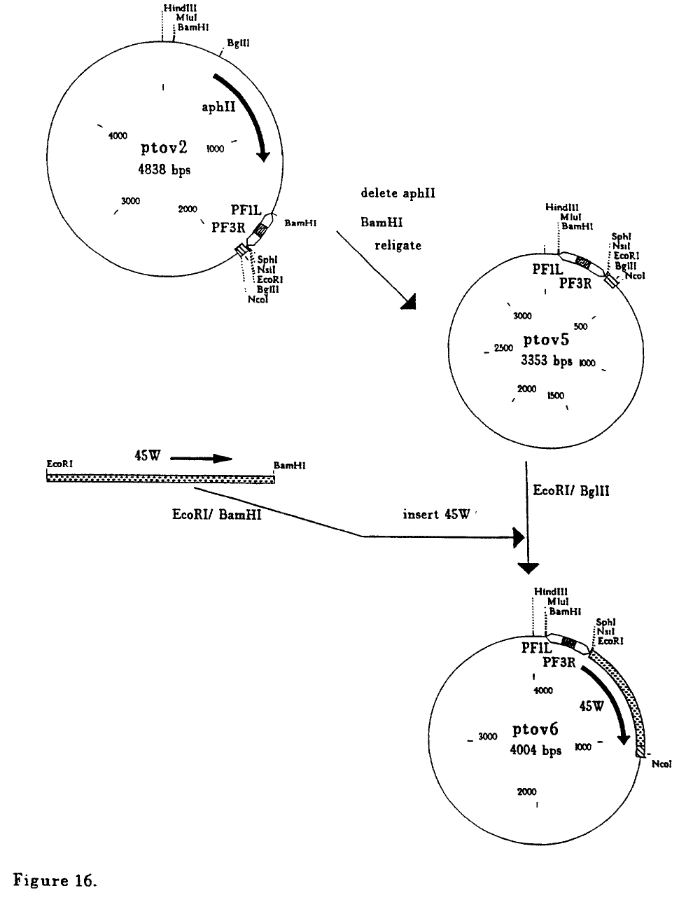

The aphII gene was removed from the plasmid ptov2 by digesting with the restriction enzymes BamHI and BglII, purifying the vector fragment and religating the free ends to form the plasmid ptov5. The DNA sequence encoding the Taenia ovis 45W antigen fragment was removed from the plasmid pGEX 45W (Johnson, K. S., Harrison, G. B. L., Lightowlers, M. W., O'Hoy, K. L., Cougle, W. G., Dempster, R. P., Lawrence, S. B., Vinton, J. G., Heath, D. D., and Rickard, M. D. (1989). Vaccination against ovine cysticercosis using a defined recombinant antigen. (Nature 338, 585-587) by digesting with the restriction enzymes EcoRI and Bam HI and ligating it into ptov5 cut with BamHI and EcoRI to form ptov6. This placed the DNA sequence encoding the 45W antigen fragment under the control of the orf virus PF3R promoter and supplied it with translational and transcriptional termination sequences. These steps are illustrated in FIG. 16.

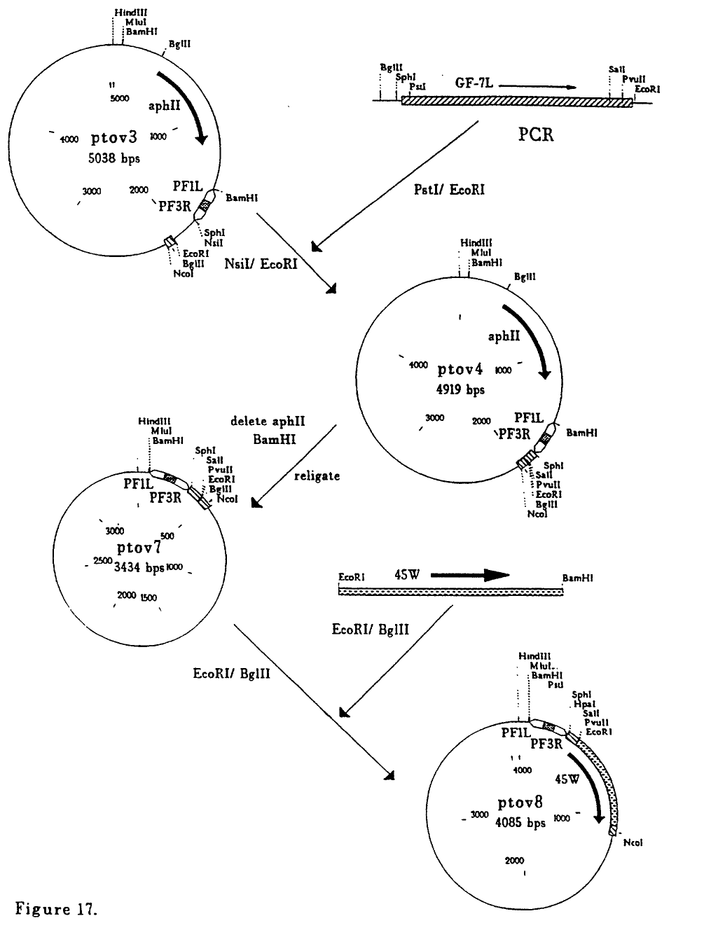

A 73 bp fragment from the 5′ portion of the VEGF-like gene from orf virus NZ-7 encoding the presumptive secretory leader sequence was amplified with specific primers which introduced a new initiation codon, a PstI and an EcoRI restriction site into the amplified DNA fragment. The amplified fragment was digested with PstI and EcoRI and cloned into ptov3 cut with NsiI and EcoRI to create the plasmid ptov4. The plasmid ptov4 was digested with BamHI to remove the aphII gene, purified by agarose gel electrophoresis and religated to form the ptov7. The DNA sequence encoding the 45W antigen fragment was removed from the plasmid pGEX 45W by digesting with the restriction enzymes EcoRI and Bam HI and ligating it into ptov7 cut with BamHI and EcoRI to form ptov8. This placed the 45W antigen fragment under the control of the orf virus PF3R promoter and supplied a 5′ protein secretory leader sequence in addition to the 3′ translational and transcriptional terminators present in ptov6. These steps are illustrated in FIG. 17.

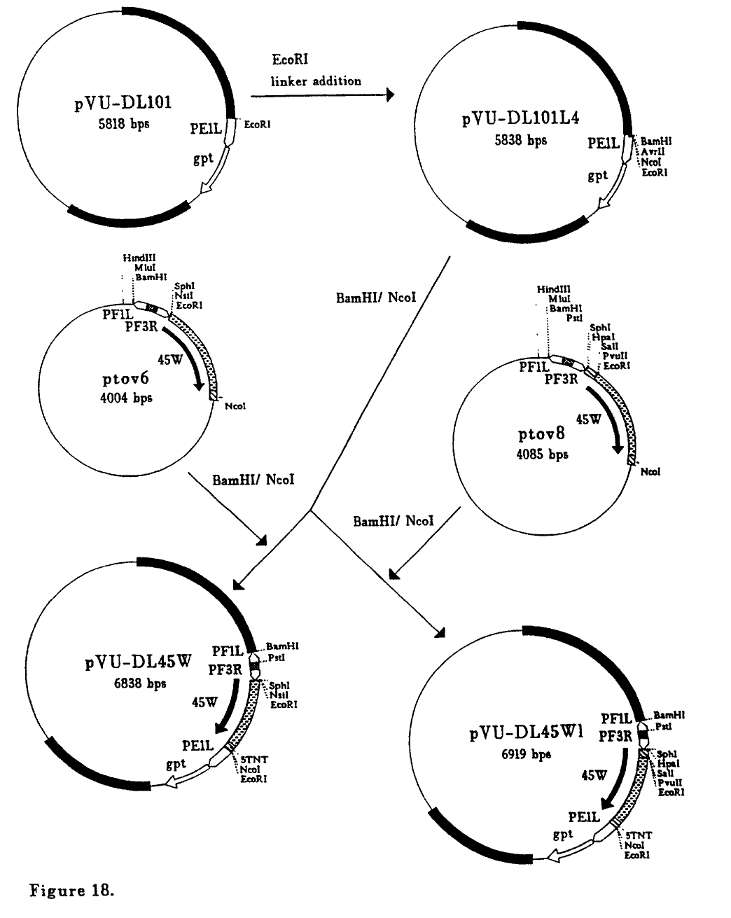

The plasmid pVU-DL101 was cut with EcoRI and an oligonucleotide linker containing a BamHI and a NcoI restriction site was ligated in to form the plasmid pVU DL101L4. This plasmid was then digested with BamHI and NcoI to allow the insertion of both versions of the chimeric 45W gene from ptov6 and from ptov8. The resulting plasmids were designated pVU-dl45W (from ptov6) and pVU-dl45W1 (from ptov8). These steps are illustrated in FIG. 18.

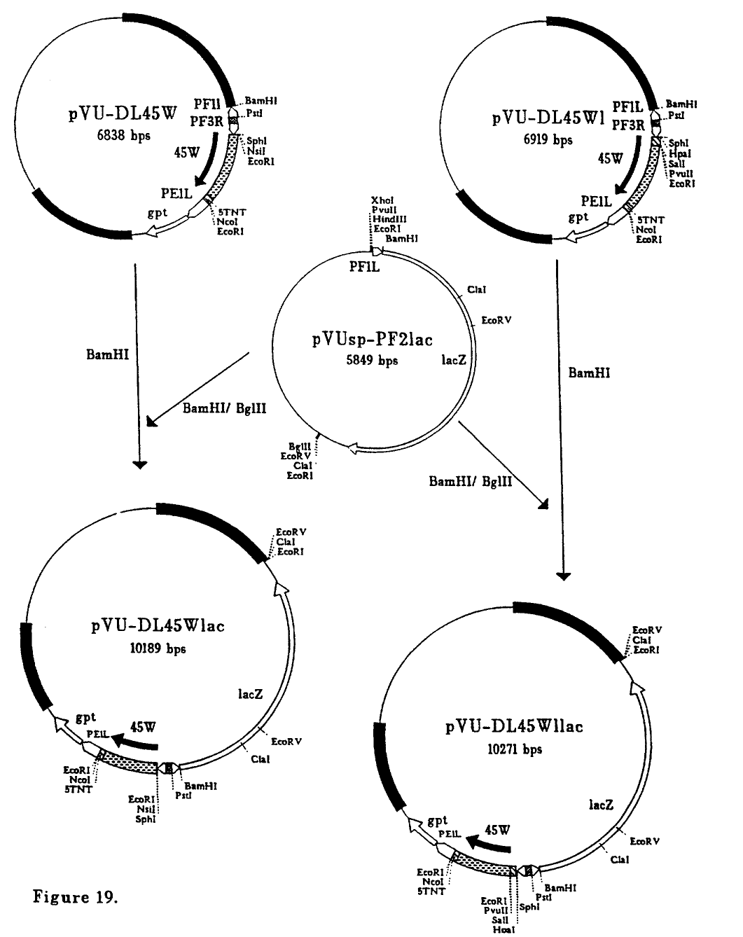

A promoterless lacz gene was cleaved out of the plasmid pVUsp-PF2lac, a derivative of pSP PFlac illustrated in FIG. 11 by digestion with BamHI and BglII. In this latter version of the plasmid, the F1L promoter fragment has been truncated to 100 base pairs and a BglII restriction site introduced distal to the lacz gene. The lacz fragment was gel purified and ligated into both pVU-DL45W and pVU-Dl45W1 at a unique BamHI site. This placed the lacz gene under the control of the F1L promoter and completed the construction of the transfer vectors for introducing the T. ovis 45W gene into the orf virus genome. These steps are illustrated in FIG. 19.

The same oligonucleotide linker containing the BamHI and a NcoI restriction sites was ligated into the plasmid pVU-DL102. This plasmid contains the x-gpt gene cloned in the opposite orientation to that in pVU-DL101 (FIG. 14). Cloning steps parallel to those described for pVU-DL101 were subsequently performed and the transfer vectors which were generated were designated pVU-DL45W6lac and pVU-DL45W8lac. These contained the same sequences as pVU-DLA5Wlac and pVU-DL45W1lac respectively, but differed in that the entire inserted region was in the opposite orientation to that illustrated for these plasmids in FIG. 19.

### EXAMPLE 8

### Transfection Protocol

Primary bovine testis (BT) cells were grown in monolayer cultures in Eagle's Minimal Essential Medium (MEM; Sigma Cat. No. M0643) supplemented with lactalbumin hydrolysate (5 g/L) and 5% foetal calf serum. Medium for selecting orf virus transformants expressing x-gpt contain mycophenolic acid, 25 μg/ml, xanthine, 250 μg/ml, hypoxanthine, 15 μg/ml, aminopterin, 1 μg/ml, thymidine, 5 μg/ml and 2% foetal calf serum. Lactalbumin hydrolysate was omitted from the selective medium and replaced with additional non-essential amino acids (MEM non-essential amino acid mixture, Sigma Cat. No. M2025).

BT cells were grown as monolayers in a suitable cell culture vessel. Twenty-four hours prior to infection, the cell growth medium was replaced with the selective medium containing mycophenolic acid. The cells were infected with orf virus, strain NZ-2, (moi 0.05-0.1) and the virus allowed to adsorb for 1 hour. Cell monolayers were washed 2 times with opti-MEM serum-free medium, (Life Technologies Inc, Gaithersburg, Md. U.S.A.) to remove residual foetal calf serum, and drained. A 1.0 ml volume of opti-MEM containing 10. μl Lipofectin reagent (Life Technologies Inc, Gaithersburg, Md., U.S.A.) and approximately 2.0 μg plasmid DNA diluted according to the suppliers instructions was added to each flask and incubated overnight. Following this overnight adsorption step, 5.0 ml of selective medium containing 2% foetal calf serum was added and the incubation continued until cytopathic effect (CPE) was observed approximately 3-5 days post-infection.

Cell monolayers were scraped from the flask, deposited in the bottom of a centrifuge tube by low speed centrifugation, washed with phosphate buffered saline (PBS) and resuspended in PBS. A suitable tissue culture vessel was seeded with BT cells to produce a confluent monolayer. Routinely, 60 mm diameter polystyrene dishes were used, seeded with 1.5×106 cells per dish and incubated in a CO₂ atmosphere to maintain a pH of around 7.2. The culture medium was removed and 0.5 ml of an appropriate dilution of orf virus in PBS was added and incubated for one hour at 37° C. Dishes were tipped at 15 min intervals to ensure an even distribution of fluid. At the end of this time the inoculum was removed and growth medium containing 1% agarose added. After five days, the time when plaques usually become visible, X-gal was added to the dish in a 1% agarose overlay and incubated a further 12 hours for colour development to occur. Single plaques are picked, resuspended in PBS and inoculated into a partially drained cell culture vessel which had been seeded with 2×105 cells and grown to confluence as described. One ml of medium was added to each well and incubation at 37° C. continued until a complete cytopathic effect was observed. The cell culture vessels were placed at −20° C. until the contents were frozen after which time they were thawed. The cell lysates were used as a source of virus, for further plaque purification, and of viral DNA for hybridisation. Viral DNA was prepared from cytoplasmic extracts of BT cells by the method of Moyer, R. W. and Graves, R. L. (1981). The mechanism of cytoplasmic orthopoxvirus DNA replication. Cell 27, 391-401. The isolated DNA was digested with restriction enzymes to confirm the insertion of the foreign genes. Frequently, the first plaque purification step fails to remove all the wild type virus and a series of plaque purification steps may be performed in order to obtain a pure culture of mutated virus. Bulk cultures of virus are grown in 150 cm2 tissue culture flasks and the virus purified by the method described in Robinson, A. J., Ellis, G. and Balassu, T. (1982). The genome of orf virus: restriction endonuclease analysis of viral DNA isolated from lesions of orf in sheep. Arch Virol. 71, 43-55. DNA is extracted from the purified virions by the method described in Balassu, T. C. and Robinson, A. J. (1987). Orf virus replication in bovine testis cells: kinetics of viral DNA, polypeptide, and infectious virus production and analysis of virion polypeptides. Arch Virol. 97, 267-281.

### EXAMPLE 9

### Assessment of Orf Virus Modification

In order to determine whether or not the viruses recovered from the transfections and plaque purifications were modified to carry the inserted genes, DNA was prepared from infected cells and tested by hybridisation by methods well known to those skilled in the art, for example, Merchlinsky, M. and Moss, B. (1989). Resolution of vaccinia virus DNA concatemer junctions requires late-gene expression. J Virol. 63, 1595-1603. In the preparation of mutated orf virus DNA for these tests, a 100 μl aliquot of orf virus-infected BT cells in PBS was centrifuged for 30 min at approximately 12,000 g. The cell pellet was resuspended in 50 μl 0.15 M NaCl, 20 mM Tris, 10 mM EDTA, pH 8.0. A 250 μl volume of 20 mM Tris, 10 mM EDTA, 0.75% SDS containing a protease at an appropriate concentration (e.g. Proteinase K at 0.5 mg/ml) was added to each sample and incubated at 37° C. for 3 hours. The samples were extracted with an equal volume of phenol:chloroform (1:1) before precipitation with ethanol. Following centrifugation the ethanol-precipitated DNA was redissolved in 50 μl TE. The material harvested from the various passages was subjected to the hybridization procedure with a specific x-gpt probe. A positive result can be obtained with pVU-DL106 for the transfection two hours post-infection as early as passage one. An alternative procedure that was used to detect heterologous DNA markers in recombinant virus was to amplify DNA sequences by the polymerase chain reaction using primers specifically designed to amplify the foreign DNA sequences. Other transfections may require further passages for the detection of recombinant viruses. Transfections performed with the plasmid pVU-DL106 at two hours allowed CPE to be detected at three days post-inoculation at passage three and the detection of mutated virus containing the x-gpt gene as determined by DNA-DNA hybridization. A qualitative assay for β-galactosidase activity using the chromogenic substrate 5-bromo-4-chloro-3-indolyl-β-D-galactoside (X-gal) was used to detect mutated orf virus containing the β-galactosidase gene.

### EXAMPLE 10

### Construction of a Vector Plasmid Suitable for the Insertion of Foreign Genes into the Region of the Orf Virus Genome Corresponding to the Orthopoxvirus ATI-Region

The intergenic region between the RNA polymerase subunit gene, rpo 132 and the open reading frame of the presumptive gene (H)I1L was identified as a suitable target site for the insertion of foreign DNA. The region is 90 nucleotides in length and lies between two converging transcriptional elements one of which, rpo 132, is an essential gene. A plasmid, PB-23ΔSal, which contains a sequence of 1.6 kilobases extending into the unsequenced region upstream of position 1 shown in the sequence illustrated in FIG. 5 and terminating at the PstI site at position 178 was used as the template in a PCR cloning reaction. A sequence of 1.0 kb was amplified from it using the primers zxs-1 GATCCCGCTCGAGAACTTCAA (forward) which is complementary to a sequence identified in PB-23ΔSal that contains an existing XhoI restriction site and zxs-2 GTCAGATCTATGCATAAAAATTTCGCATCAGTCGAGATA (reverse) which introduces a BglII, a NsiI and an ApoI restriction site. The amplified fragment was purified by electrophoresis on a 1% agarose gel and digested with the restriction enzymes XhoI and BglII. The purified fragment was ligated then into the plasmid pSP-70 at the corresponding XhoI and BglII sites creating the plasmid pTvec1. This cloning step also introduced a poxvirus transcriptional termination signal (5TNT) into the vector.

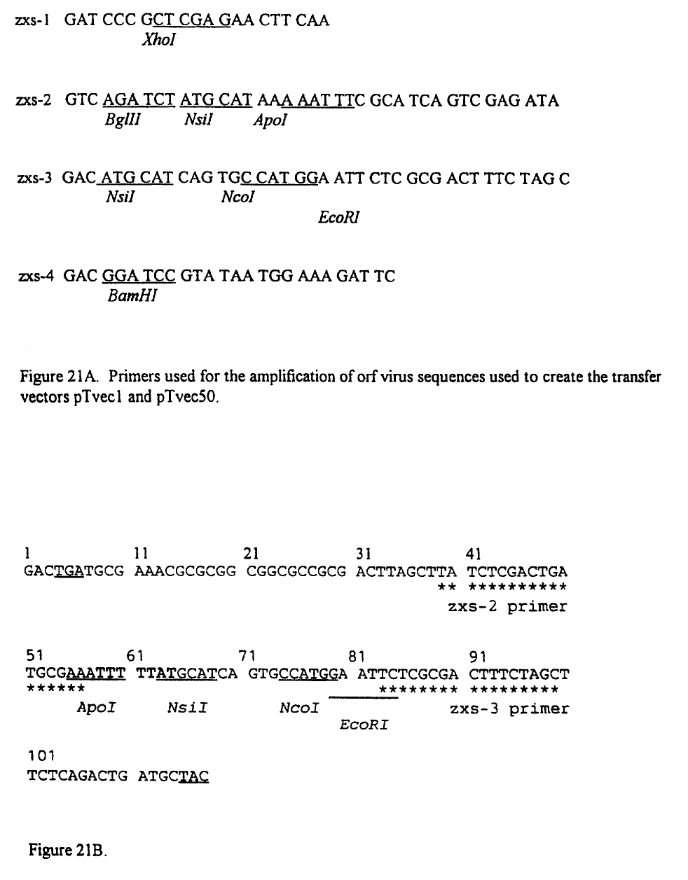

A second fragment comprising the sequence located between nucleotide positions 66 and 1069 (FIG. 5) was amplified with the primers zxs-3 GACATGCATCAGTGCCATGGAATTCTCGCGACTTTCTAGC (forward) which introduces NsiI, NcoI and EcoRI restriction sites and zxs-4 GACGGATCCGTATAATGGAAAGATTC (reverse) which introduces a BamHI restriction site. The amplified fragment was digested with the restriction endonucleases BamHI and NsiI and purified in the same manner as the first fragment. The purified fragment was then cloned into pTvec1 which had been cut with NsiI and BglII. The resulting plasmid pTvec50 contains a series of restriction sites and a transcriptional termination signal which are available for further cloning steps. These restriction sites are ApoI, NsiI, NcoI and EcoRI. The sequence of the primers, the restriction sites and the sequence of the modified intergenic region are shown in FIGS. 20A and 20B. The cloning steps involve in the construction of ptvec50 are illustrated in FIG. 21.

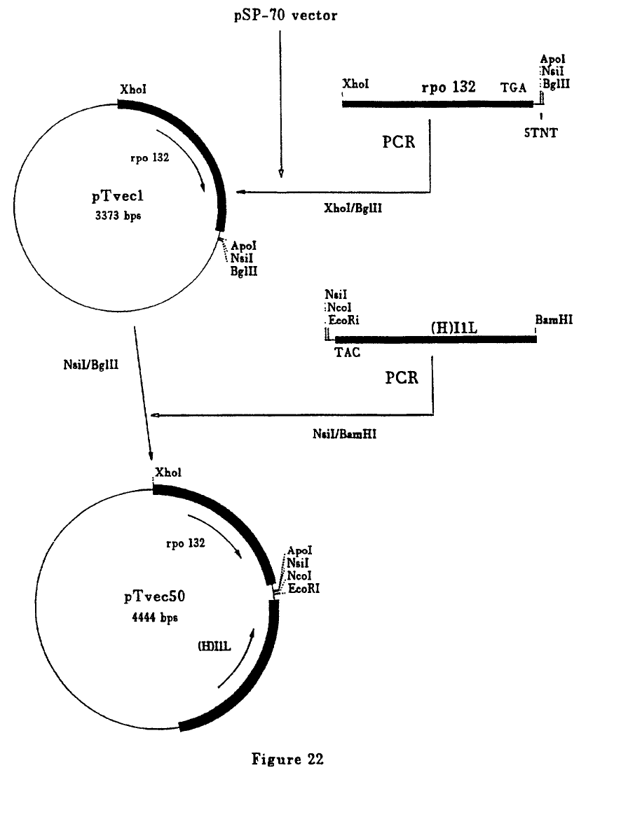

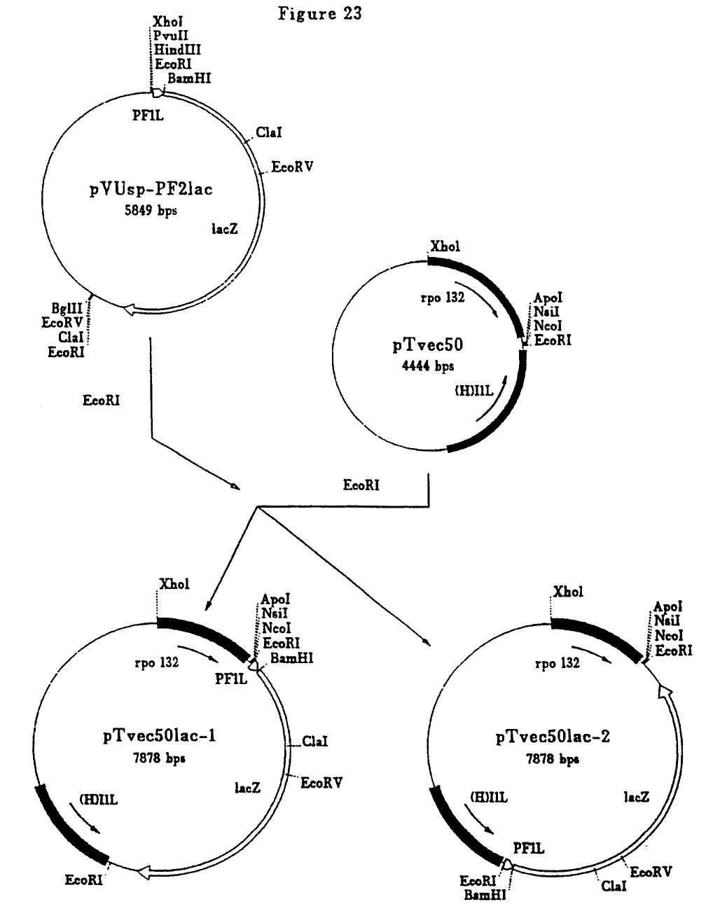

A lacz gene under the control the orf virus late promoter PF1L was cleaved out of the plasmid pVUsp-PF2lac with EcoRI. The fragment was gel purified and ligated into the EcoRI site of pTvec50. Recombinant plasmids containing the lacz gene in both possible orientations were recovered and designated pTvec50lac-1 and pTvec50lac-2. The cloning steps involved in the construction of pTvec50lac-1 and pTvec50lac-2 are illustrated in FIG. 22. This completed the construction of a transfer vector designed to introduce the foreign gene lacz into the intergenic site between the open reading frames of rpo 132 and (H)I1L shown in FIG. 5.

In this example the xgpt gene was not included in the transfer vector and consequently selection of recombinant orf virus expressing xgpt by growth in the presence of mycophenolic acid was not able to be used as a selection method. Virus recombinants were selected by using lacz expression as the primary method for identifying recombinants containing an insertion in the ATI region. The following variation of the method described in Example 8 was used.

Primary bovine testis (BT) cells were grown in monolayer cultures in Eagle's Minimal Essential Medium (MEM); (Sigma Cat. No. M0643) supplemented with lactalbumin hydrolysate (5 g/L) and 5% foetal calf serum. Prior to infection the cell growth medium was removed and the cells washed briefly with phosphate buffered saline (PBS) to remove residual serum. The cells were infected with orf virus, strain NZ-2, (moi 0.05-0.1) and the virus allowed to adsorb for 1 hour. Cell monolayers were washed 2 times with opti-MEM serum-free medium, (Life Technologies Inc, Gaithersburg, Md., U.S.A.) to remove non-adsorbed virus and residual foetal calf serum, and drained. A 1.0 ml volume of opti-MEM containing 10 μl Lipofectin reagent (Life Technologies Inc, Gaithersburg, Md., U.S.A.) and approximately 2.0 μg plasmid DNA diluted according to the suppliers instructions was added to each flask and incubated overnight. Following this overnight adsorption step, 5.0 ml of selective medium containing 2% foetal calf serum was added and the incubation continued until cytopathic effect (CPE) was observed approximately 3-5 days post-infection.

Cell monolayers were scraped from the flask, deposited in the bottom of a centrifuge tube by low speed centrifugation, washed with PBS and resuspended in PBS. The resuspended cells were subjected to three cycles of freezing and thawing and sonicated briefly. The virus titre of the harvested culture was determined and the material plated on fresh dishes of BT cells at a dilution calculated to give approximately 2000 virus plaques per dish. Sufficient material was plated to screen 50,000 plaques (25 dishes). The infected monolayers were grown under an a 1% agarose overlay and after 5 days incubation when plaques became visible, X-gal in a 1% agarose overlay was added to the dishes and incubated a further 12 hours for colour development to occur. At this stage, any coloured plaques which had appeared were picked and treated as described in Example 8. Further purification of the recombinant virus was achieved by repeated cycles of plating and picking single, coloured plaques until a pure culture of lacz positive virus was obtained.

### APPLICATION OF THE INVENTION

In accordance with the present invention there is provided a parapoxvirus vector, specifically an orf virus vector, containing exogenous DNA. The exogenous DNA may encode an antigen capable of inducing an immune response or may encode a heterologous polypeptide of which expression is desired.

The vectors of the present invention therefore have particular applications in the expression of heterologous polypeptides and antigens. The capacity to express antigens make these vectors particularly suitable for use in vaccines.

Orf virus vectors have a number of advantages over vaccinia virus vectors. Orf virus has a relatively narrow host range compared to vaccinia. This reduces the vaccinia associated risks of cross-species infection and spread of disease. A further advantage is that orf virus is less virulent than vaccinia in man, reducing the risks of febrile response and lesions.

It will be appreciated that the above description is provided by way of example only and that the invention is limited only by the scope of the appended claims.

### References

Balassu, T. C. and Robinson, A. J. (1987). Orf virus replication in bovine testis cells: kinetics of viral DNA, polypeptide, and infectious virus production and analysis of virion polypeptides. Arch Virol. 97, 267-81.

Beck, E., Ludwig, A., Aurswald, E. A., Reiss, B. and Schaller, H. (1982). Nucleotide sequence and exact location of the neomycin phosphotransferase from transposon Tn5. Gene. 19, 327-336.

Boyle, D. B. and Coupar, B. E. (1988). Construction of recombinant fowlpox viruses as vectors for poultry vaccines. Virus Res. 10, 343-56.

Brosius, J. (1989) Superlinkers in cloning and expression vectors. DNA 8 759-777

Cochran, M. A., Mackett, M. and Moss, B. (1985). Eukaryotic transient expression system dependent on transcription factors and regulatory DNA sequences of vaccinia virus. Proc Natl Acad Sci U.S.A. 82, 19-23.

Falkner, F. G. and Moss, B. (1988). Escherichia coli gpt gene provides dominant selection for vaccinia virus open reading frame expression vectors. J Virol. 62, 1849-54.

Felgner, P. L., Gadek, T. R., Holm, M., Roman, R., Chan, H. W., Wenz, M., Northrop, J. P., Ringold, G. M. and Danielsen, M. (1987). Lipofection: a highly efficient, lipid-mediated DNA-transfection procedure. Proc Natl Acad Sci USA. 84, 7413-7417.

Fleming, S. B., Fraser, K. M., Mercer, A. and Robinson, A. J. (1991). Vaccinia virus-like early transcriptional control sequences flank an early gene in the orf parapoxvirus. Gene. 97, 207-212.

Fleming, S. B., Blok, J., Fraser, K. M., Mercer, A. A. and Robinson, A. A. (1993). Conservation of gene structure and arrangement between vaccinia virus and orf virus. Virology. 195, 175-184

Fleming, S. B., Lyttle, D. J., Sullivan, J. T., Mercer, A. A. and Robinson, A. J. (1995) Genomic analysis of a transposition-deletion variant of orf virus reveals a 3.3 kbp region of non-essential DNA J Gen Virol. 76 2969-2978

Fraser, K. M., Hill, D. F., Mercer, A. A. and Robinson, A. J. (1990). Sequence analysis of the inverted terminal repetition in the genome of the parapoxvirus, orf virus. Virology. 176, 379-89.

Graham, F. L. and Van der Eb, A. J. (1973). A new technique for the assay of infectivity of human adenovirus type 5 DNA. Virology 52, 456-467.

Johnson, K. S., Harrison, G. B. L., Lightowlers, M. W., O'Hoy, K. L., Cougle, W. G., Dempster, R. P., Lawrence, S. B., Vinton, J. G., Heath, D. D., and Rickard, M. D. (1989) Vaccination against ovine cysticercosis using a defined recombinant antigen. Nature 338 585-587.

Lyttle, D. J., Fraser, K. M., Fleming, S. B., Mercer, A. A. and Robinson, A. J. (1993) Homologs of vascular endothelial growth factor are encoded by the poxvirus orf virus. J. Virol. 68 84-92

McGeoch, D. J. (1990). Protein sequence comparisons show that the ‘pseudoproteases’ encoded by poxviruses and certain retroviruses belong to the deoxyuridine triphosphatase family. Nucleic Acids Res. 18, 4105-10.

Mead, D. A., Szczesna-Skorupa, E. and Kemper, B. (1986). Single-stranded DNA “blue” T7 promoter plasmids: a versatile tandem promoter system for cloning and protein engineering. Protein Eng. 1, 67-74.

Melton, D. A., P. A., K., Rebagliati, M. R., Maniatis, T., Zinn, K. and Green, M. R. (1984). Efficient in vitro synthesis of biologically active RNA and RNA hybridization probes from plasmids containing bacteriophage SP6 promoter. Nucleic Acids Res. 12, 7035-7056.

Mercer, A. A., Fraser, K., Barns, G. and Robinson, A. J. (1987). The structure and cloning of orf virus DNA. Virology. 157, 1-12.

Mercer, A. A., Fraser, K. M., Stockwell, P. A. and Robinson, A. J. (1989). A homologue of retroviral pseudoproteases in the parapoxvirus, orf virus. Virology. 172, 665-8.

Merchlinsky, M. and Moss, B. (1989). Resolution of vaccinia virus DNA concatemer junctions requires late-gene expression. J Virol. 63, 1595-603.

Miller, J. H. (1972). “Experiments in Molecular Genetics.” Cold Spring Harbor Laboratory, Cold Spring Harbor, N.Y.

Moss, B. (1990). Regulation of vaccinia virus transcription. Annu Rev Biochem. 59, 661-88.

Moss, B. (1990). “Poxviridae and their replication.” in Virology, B. N. Fields, D. M. Knipe, R. M. Chanock, M. S. Hirsch, J. L. Melnick, T. P. Monath and B. Roizman, 2nd. Raven Press, New York, 2079-2111.

Moyer, R. W. and Graves, R. L. (1981). The mechanism of cytoplasmic orthopoxvirus DNA replication. Cell. 27, 391-401.

Mulligan, R. C. and Berg, P. (1980). Expression of a bacterial gene in mammalian cells. Science. 209, 1422-1427.

Mulligan, R. C. and Berg, P. (1981). Selection for animal cells that express the Escherichia coli gene coding for xanthine-guanine phosphoribosyl transferase. Proc Natl Acad Sci USA. 78, 2072-2076.

Naase, M., Nicholson, B. H., Fraser, K. M., Mercer, A. A. and Robinson, A. J. (1991). An orf virus sequence showing homology to the fusion protein gene of vaccinia virus. J Gen Virol. 72, 1177-1181.

Robinson, A. J., Ellis, G. and Balassu, T. (1982). The genome of orf virus: restriction endonuclease analysis of viral DNA isolated from lesions of orf in sheep. Arch Virol. 71, 43-55.

Robinson, A. J., Barns, G., Fraser, K., Carpenter, E. and Mercer, A. A. (1987). Conservation and variation in orf virus genomes. Virology. 157, 13-23.

Robinson, A. J. and Balassu, T. C. (1981) Contagious pustular dermatitis (orf). Vet Bull 51, 771-761

Robinson, A. J. and Lyttle, D. J. (1992) “Parapoxviruses: their biology and potential as recombinant vaccines.” in Recombinant Poxviruses, Chapter 9, 306-317 eds M.Binns and G. Smith CRC Press, (1992), Boca Raton.

Sambrook, J. Fritsch, E. F. and Maniatis, T., Molecular Cloning: A Laboratory Manual (Second Edition), Cold Spring Harbour Laboratory Press 1989.

Sullivan, J. T., Fraser, K., Fleming, S. B., Robinson, A. J. and Mercer, A. A. (1995) Sequence and transcriptional analysis of an orf virus gene encoding ankyrin-like repeat sequences. Virus Genes, 9, 277-282

Viera, J. and Messing, J. (1982). The puc plasmids, an M13mp7 derived system for insertion mutagenesis and sequencing with synthetic universal primers. Gene. 19, 259-268.

Vogelstein, B. and Gillespie, D. (1979). Preparation and analytical purification of DNA from agarose. Proc Natl Acad Sci USA. 76, 615-619.

Weinstock, G. M., Berman, M. L. and Silhavy, T. J. (1983). “Chimeric genetics with β-galactosidase in gene amplification and analysis.” in Expression of Cloned Genes in Procaryotic and Eucaryotic Cells, T. S. Papas, M. Rosenberg and J. A. Chirikjian, Elsevier, New York, 27-64.

1. A parapoxvirus vector comprising a parapox virus containing exogenous DNA.

2. A vector as claimed in claim 1 wherein the parapox virus is orf virus.

3. A vector as claimed in claim 1 or claim 2 wherein the exogenous DNA encodes at least one gene product.

4. A vector as claimed in claim 3 wherein one gene product encoded is an antigen capable of inducing an immune response.

5. A vector as claimed in claim 4 wherein the antigen is selected from the group consisting of HIV envelope protein, herpes simplex virus glycoprotein, Taenia ovis, Echinococcus granulosis antigens, Trichostronglylus antigens, Haemonchus antigens, Ostertagia antigens and combinations thereof.

6. A vector as claimed in claim 5 wherein the antigen is a Taenia ovis antigen selected from the group comprising Taenia ovis 45W, 16 kd, 18 kd antigens and combinations thereof.

7. A vector as claimed in any one of claims 3 to 6 wherein the exogenous DNA further encodes at least one product which is a biological effector molecule.

8. A vector as claimed in claim 7 wherein the biological effector molecule is selected from the group comprising γ interferon, IL-1, IL-2, IL-1β, IL4, IL-5, IL-6, IL-12 and combinations thereof.

9. A vector as claimed in claim 8 wherein the biological effector molecule is selected from the group comprising IL-1, IL-2, IL-12 and combinations thereof.

10. A vector as claimed in any one of claims 3 to 9 wherein the exogenous DNA further encodes at least one peptide moiety expressed as a hybrid or chimeric protein with a native virus protein.

11. A vector as claimed in any one of claims 1 to 10 wherein the exogenous DNA is incorporated in one or more non-essential regions of the virus genome.

12. A vector as claimed in claim 11 wherein the non-essential regions are selected from the non-essential regions identified in accompanying FIGS. 2, 3, 5 and 7.

13. A vector as claimed in claim 11 or claim 12 wherein the non-essential region is from nucleic acids 11 to 16 in the sequence of FIG. 5 or from nucleic acids 2226 to 2286 in the sequence of FIG. 9.

14. A vector as claimed in any one of claims 1 to 13 wherein the exogenous DNA is under the control of a poxvirus promoter.

15. A vector as claimed in claim 14 wherein the poxvirus promoter is an orf virus promoter.

16. A vector as claimed in claim 15 wherein the orf virus promoter is selected from the As group consisting of E1L, F1L and F3L as set forth in FIG. 10.

17. A vector as claimed in any one of claims 3 to 16 wherein the exogenous DNA further encodes a reporter gene.

18. A vector as claimed in any one of claims 3 to 17 wherein the exogenous DNA further encodes a selectable marker.

19. A fragment or variant of a vector as claimed in any one of claims 4 to 18 having equivalent immunological activity thereto.

20. A vaccine comprising a viral vector according to any one of claims 1 to 18 or a fragment or variant thereof as claimed in claim 19.

21. A vaccine as claimed in claim 20 which further comprises a pharmaceutically acceptable carrier and/or adjuvant therefor.

22. A host cell incorporating a vector as claimed in any one of claims 1 to 18.

23. A host cell according to claim 22 which is a eukaryotic cell.

24. A host cell according to claim 22 or claim 23 which is a bovine testis cell or an ovine testis cell.

25. A method for producing recombinant parapoxvirus vectors comprising transfecting a vector of any one of claims 1 to 18 into a selected host cell infected with orf virus; selecting a recombinant virus; and optionally purifying the selected virus.


SEQUENCE LISTING (26 sequences)

SEQ ID NO: 1 - DNA, 1447 bp, Orf virus strain NZ-2
gggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgccgcactctcctgagcccacgcccgcggcgccgggctcgctgtacgacgtcttcctcgcgcgcttcctgcgccggctggccgcgcgcgcggcgccggcctcggccgcctgcgccgtgcgcgtgggtgcggtgcgcggccgcctgcggaactgcgagctagtggtgctgaaccgctgccacgcggacgcggccggcgcgctcgcgctagcctccgcggcgctcgccgagacgctggcggagctcccgcgcgcggacaagctcgccgtcgcgctcgagctgggcgtggaccccgagcacccggagctgacgccggaccccgcctgcgcaggcgagagcgcactcgcacagaacatcgacatccagacgctggacctgggcgactgcggagaccccaaaggccgccgactgcgcgtggcgctggtgaacagcggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtgacggtgctcggcgtggtggcggtttcactgctgcggcgcgcgctgcgggtacgctaccgcttcgcgcggccggccgcgctgcgcgcgtagccgcgcaaaatgtaaattataacgcccaacttttaagggtgaggcgccatgaagttgctcgtcggcatactagtagccgtgtgcttgcaccagtatctgctgaacgcggacagcaacacgaaaggatggtccgaagtgctgaaaggcagcgagtgcaagcctaggccgattgttgttcctgtaagcgagacgcacccagagctgacttctcagcggttcaacccgccgtgtgtcacgttgatgcgatgcggcgggtgctgcaacgacgagagcttggaatgcgtccccacggaagaagtaaacgtgacgatggaactcctgggggcgtcgggctccggtagtaacgggatgcaacgtctgagcttcgtagagcataagaaatgcgattgtagaccacgattcacaaccacgccaccgacgaccacaaggccgcccagaagacgccgctagaactttttatggaccgcagatccaaacgatgatgcgatcaggtcatgcggaagaaggcgccacggagcaaagtgaaaaaggaccgcctagcagtcgagaccctcccgccgcagccgcggacaccccacacccgccttccacccgccagacgccaacaccgcagccaacaagcatcg

SEQ ID NO: 2 - DNA, 2489 bp, Orf virus strain NZ-7
gggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtgttctcccgctggataaatgccgctgcagacgcccccgacgccgcactctcctgagcccacgcccgcggcgccgggctcgctgtacgacgtcttcctcgcgcgcttcctgcgccggctggccgcgcgcgcggcgccggcctcggccgcctgcgccgtgcgcgtgggtgcggtgcgcggtcgcctgcggaactgcgagctggtggtgctgaaccgctgccacgcggacgccggccggcgcgctcgcgctggcctccgcggcgctggcggaaacgctggcggagctgccgcgcgcggacaagctcgccgtcgcgcgcgagctaggcgtggaccccgagcacccggagctgacgccggaccccgcctgcgcgggcgagagcgcgctcgcacagaacatcgacatccagacgctggacctaggcgactgcggcgaccccaaaggccgccgactgcgcgtggcgctggtgaacagcggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtggcggtgctcggcgtggtggcaatttcgctgctgcgccgcgcgctaagaatacggtttagatactcaaagtctatccagacacttagagtgtaactttgagtaaaaaatgtaaatactaacgccaaaatttcgatagttgttaagcaatatataacatttttaaaacgtcatcaccagcatgaagttaacagctacgttacaagttgttgttgcattgttaatatgtatgtataatttgccagaatgcgtgtctcagagtaatgattcacctccttcaaccaatgactggatgcgtacactagacaaaagtggttgtaaacctagagatactgttgtttatttgggagaagaatatccagaaagcactaacctacaatataatccccggtgcgtaactgttaaacgatgcagtggttgctgtaacggtgacggtcaaatatgtacagcggttgaaacaagaaatacaactgtaacagtttcagtaaccggcgtgtctagttcgtctggtactaatagtggtgtatctactaaccttcaaagaataagtgttacagaacacacaaagtgcgattgtattggtagaacaacgacaacacctacgaccactagggaacctagacgataactaataacaaaaaatgtttatttttgtaaatacttaattattacacactttacaataatctcaaaaataaattgcgtgcccggacggctgcagctggtgacgctgctgtgtcacacactgcgtattcgattcaagttcactaacgccactaaactagttgtgcgtgtccgagtgttaaccgtacgtcaaactaacatcttacctgtccgtgacaagaactaaaacttgaaccacatatttttaaagtatatttaacaaaatcactcacactcacacaatcataaacaccacaaccacaaccaaacacgcatgagaattaatattcttacttatccgtaacactctatgctgtacatcaacgcatcagagcagtctgagtctgactaatggcggcaaacgggaacgcaggcgcgacataatcactgagaatctccgcagcaaccgctcaaggacatctctagcgctaacggctgtttgtcattcccccgtgtgttcatctcacacgacattgtgaccgtcgcaaagcacacattcaaagtgccgcatgtggaagaattcaccgtcgagacacacaccataattaaacaagatcagtgcataagagagattagcattctacagcacaccacgtgcgaatacggacctcgtaattgtttagactagaacacctctggtctaaacaacatgtccgatcttagaacagagtttatgacgcatatgtaactgtgttctttatgtagaagttatcttttatgtcactcccttgtcttagatgagttatacatgacatgatgtatgtgtcgcccgcggcggcgcggggcgctcggcggcggggctgctgcgcgcggcgggcccgcggtggcggcggctggcgcggcgctgcggccgcgggcgcgcggcggggtagcggcccgcccgcccgggcgcccgccgcagcccttgccccggaccaggcgccacggagcaaagtgaaaaaggaccgcctagcagtcgagaccctcccgccgcagccgcgacaccccacacccgccttccacccgccagacgccaacaccacagccaacaagcatgcacccctcgccgcgcaggctgctcggcgcgctcgcgtggtggcgctgggcttcctcctcggcgg

SEQ ID NO: 3 - DNA, 4051 bp, Orf virus strain NZ-2
gactgatgcgaaacgcgcggcggcgccgcgacttagcttatctcgactgatgcgaacgcgcgacctctcgcgactttctagcttctcagactgatgctaccatatcgcggcgtgctggccccaccaccagggcttctcgccgtggctgacgcggggctggctgcgacgcgcgctgcagtagctgcgcgcgccccagtcgccgcgcacgtgcgccgggggcaggctcccgtccagcgcgtgccgcgtcacctcggcgccgggccggcggcacgtgtgcacgtccgtcttgttggagacgagcaccgcgtactgccgcatggtctctatgtgatgctccaagtgcttgcccgccatccggttggactcgcagcacgtttttgcttcggctaaggttttttctagaggggatagtagcttatccacgcgctcgggcaggacgcacgcggagccgtcgaaccctactttgaacggggtcaccttgatgttcccgtcgtagcggtcccacagcatcctgaggtaggttgtaccgtcggggtctgggtctgtccacactctaagcttttcgctacagcggccgtcgtacgtaagacggtctctacgctcgtagtagtttctgcttatgttgttggggtctccatgctcgtagtagtataaatcgtacgcgcctggcttttttaagtcgttttcgtcgttgctgacgtgtatcacgtcgggataataggatatcctaactgcactacaatctatagtatttggtctagtaagctgttcgagatcaccttgttcatcatgatctactgatttgtacacggcaccgtcgtgttccgacggacgtatgaatatgtccatggtaaacgatgtacccactttggaaaacgtatcccatgcagtaaagcatagtccgtccattataaactcaggaacactcataacaaatcgaaatctgtgaagtttttcgaacaccacttttacatggtctttgtcacgaacatcattgccgtttacttcagacatgaattgaaggaacgctaaagagtttcttgtttcttcatgaatctttccattatacgtccatccagtttctagaattctatatatgctttttgcatcgaccccgtaccaccagtacatgggaactccgaaatatatagctgggtttgagtaccaatgggcaagagtgcccattgcgtttaaaaagtcttgacaaaaaaatgcagtttttctgtcgataacttgacttggactacgttcgtggacatcgtacatgtccataattggttcattggtaacggttacatgacccgtcattatctttttaacaatcataagatacagtttgcctaaagtcgaaatatgtataacgttaatttttacatgttctcctaacgtaattgcgtttttacttagccattcgtcgtctacaaaaatcttacgatacataggatttctctctacgtatcttctaaagtatagatttaccggtctaccggcgacattagcgccatctatagcaggagcaagctgtatgtatcgtcgtataatgtctcgtataagctttctgtcttctctgggaatacacgacacggaacttagagactggtgccagtgtctttcaaccaaagacttgaacctagcaaccaacgcgttgtcactctccatttataattaaataattatcccaacttcgtatgttaatccttattaccagatagcaccgctccttcctctccaccacgtactatctaaaggatacctgtaagggtaatgtctggataacgggcgtgtgagccaagacgtgttatggtgtcttccccaccaacggtccacttctctaactaccggagtgctagacgttgtcgatcccactactgttgtttctccattacctgtaatctttgaagcgcaacaagtgttagtctttgccaaatcttctaacggcttaattaggtcgttaagtctgtcaatatccatgcactgaggtgtatcgttaccagtttccccaactttgggaggaacttcctgtttagtgtccaaataacccataaacctgtctctaagcattcttctgtacgtgcttttgtcattgtctatgtctcccaaaaacctgtgatttacgcatccgttgtacgtaagtctgtcccttcgctctcgctcgttatttcctacttcttctattgtactgtaatgttgatagtccaagtaatagccactgttttcatgatttcttgtaaatataatcggtgttttattattgacatcgtgttgcctactgtacgtatcttccatggatctaggaacttgtctagaaaattgaggactagaaatacgccttccaaatcctggatgataaaccaaacgcaatgcactacagtcgacatcgtcactgtctctagttatcccatcaacaactccttctttgtgatgctccactgttttaaagttaactcctctgtatatgttgagtgttaaaaaaatatccacattgaacgcagttccatattttgttattttatcccacgacgtataacacaaaccatccataatgaattccggaaccgatactacaaaattagtatcgaacttatcaaacgaaatgtttactcgcggttgtgcttcgtattttttgtcatacacatctgtcatgtattgtacaaaatctatagctcctctagcatcgggcatgctaacgtctggaaaatcattttttaactgttctagtacgaactgttgattttggtcatctctccaccagtacattggcaatcctatcacaagagacttatttttataataatttgctacagaagctatatgccacatgaaattatagcaaaaataatccatctgtatgtttaaaaccggtttactagtctgctgagagtagctatccatgatagtgtttccctcgccaatacttcctgacattattctgtaaacaaccattaacaaaaatcttcctaccgttgttatgttgtcaaaagttcttatgttttgagcactttcgagtaaccatatgttattttcaaatatacttttgtaaactggatttctgtccacataactctttaaaaatagatttactggaaggccgcttgggttatctccctgtataggcggcgcttgctttatgtattcgcgtaacaaatctcttacaacttttctagttcttctaggtatacatgacctattattcaaaggctttgtccagttagcgtttataaacgttgtaaagtcactgactagcttctccatttataattaaataattacagacggcaacacagcggttatctaatatctgctgtatcctgtctgtacatctatttttctgttgagatcaagaagagctttacgtagactctccaagtgtctttctagtctgtctaaccggttacctgtttctctgcagcaatcagttatagttttgtaactgtctaacaagcttacgaggcgctcttccacactttctttagttggagctccagccgcgtacactccgttggttgaattgcctgtatcatcaggctgagtcaataggttttctccgtcattttcatccatattgagtccaacgaacacaaacgagtaagtgttcctctatttaaagtattgattttagaaaaaggcaagcctcgctgccctgattcggcggcaaacacgggttgaacacgcggaagtcgctcgcggccgtgaagatctcgtccgcgcacgcctccacgctcgcgaagcgcccaggcgagacgccgtcgtgcgagcggaacccgaactccgaggccgccaccgccgcgcccttgaagagcacgcagcgccacttcttgcgcacgtcgaaggcctcgtcgttggggtcgaacacgcgccggtccacgcgcgggccgcccgccgtgcgcgcgaactccagcgccgcgttcgccgcgttgaactcgcggatgttgtcgtagttctcgtagacggcccagagctgcagcgccacgaacatggcggccgccgccgcgagggccacgcagagcgcggacaccgcgtccatcttttatgtgcagaattattcgttggc

SEQ ID NO: 4 - DNA, 4372 bp, Orf virus strain NZ-2
ggatccatttagttattaaaattaatcatatacaactcttttatggcggctatggattcggctatccagtccttgaccgagcccacgatgcccgccaggaacaggaagaaggcgaactccaggtccacgcggttcagagagtcgctgaagtacacgaagacgtcgctgtccgggaagaagctgcgccggaacatgttgtacccgttgaccttgtgcgcgacgtgctccgcgctcagcagcgtctcgtcgaaggggtacgggtcgctgaagcggaacacgtacatggccgggtttgcgtagtagtacttcatggtgtttgtgacgaagaggctcgccagcgagatgatgatttttttcttctcgatctcgatcttgatgtggtcctcgaagcgcttcatgttgtaggcgttggtgtcgtgcacgcggatgagcacgcgcgagtccgacatgatgtcctggaactccgcgcgcgcgtcggggctctcggcgggcgtctccgcgggccgcgccacctccgcgcacaccgtcggcctagcgcgcggcggcgtgcgcatgggccgcgcccccacgcgctgcgaagcgaaaaactccacggcgcgagcctcgcccgcgtccgcgtacgactccaccaggtagttgcggctgcgcgtggtgcggccgatggtgttcagccggtgcagctccgcgaccagccggcggtagtgcgcctccagctcctcgggcatgatggaggtgtacacctcggtgagcagcatcacggtgtcgaagtcctccttgccgcagacgcgcgtcttcacgaggaagtggtgcacagccgtcgcgatagagagccgcagcgtggactcggtgacctcgacgctggcgtccttggtcttcttcgcgctccgcgaggccatgaacgagacgaggaagtccgcgctgctgttgagcacgatgaccagcgcgacgatgaagttgaggttcagcgtcttcgcggactggaacagctcggtggccgacgcgtgcacgtcgagcaggttcgcggagagccgcaggaagaacacgccgcgcttgatctcggccgcgaagcgacgttcgtactcctgccggcgcgcgttgatcgcgatgaggaagttcaggatgagccggttgatgttgtacttcacggcccaggtctgcgtcttcatgatggtgtcgaaggacatcacgatgttgaagatgaagcgctggctgtgcgagaagtagctgtagggctcgctgaggaagatggacttgttggtcgcgggcaccaccacgcccgcgcgcgcgccggacgcgtcggtgttcaggtccgggatgttcatgccgcagatgcggcagtaggccatgccgtcctcaaagtacacgaactcctccacgaactcgttgatcttggcgaagtagtccacgtccacgcgcatcgcgaccgcgagccggatctggtgctcgcagggcggcgactcgaagcgcaccccctcgccccagcccggcggctcgcgcacgaccagcgcggtgcgcgaggccgggcggaacttggcgtcgcgcgcgttgagcagcgccgggaagaggtcgcagaggtgccggctcgagaggaacacgtacttgtacagcagccggcgcgcgtccgcggccatggcgtccacgaaggcgcggccccactccgcgaccgcgggctgctcctccgcaaagttgttcgggtagaccttgtccgtggccgcgaggaacaccttcttcacgtcgaggaagtcgcggatcacgatggggacgcgcgcgccgtcgagctcgtacatgaacacgtagcgcaggttgagcttgcgccgcgagaccgggatgccgatgtgccgacacaggtacgcgaactcgaggtacttcttcgagaagcggatgcggtccaggttcttggagacgtactgcagcatgttgcgcatgttgaaggggatctcgcgcacggcgggctccgcggcgtcgtcgaaggcggtgcgcagatcgctggtgcgctgtacgaccacggcttcgccggtggcgtcgtcgtgcaccagcacgttaacgcgccgctgccggatgaccatgtcgaaggtgttgaagaacatctcgtacatgctgtgccgagtgtcgtccgcgatgcgctcgcccaccgagaggctcgcggtggcgtcgtcacgcacctgcttctcgaacttgtacccgatgtaggagaatatcgagatcagcgtggcgtcgtcggcgtcggggttctgctccatggtcgcgaagagcaggcggatgtcgtcctccgtgatcgcgtccacgttgtacaggttgaccacgaagatggacttgttctcggcgatgaagtccgtgtaggacttggtggccgtgttcgggtcgcgcatgtacgcgcggatcttcggcacgatgctcgcgaggatggactccctggaatccatttaaggacggcaagggcgcgcgagaccgtctcaaaactgaaatcgtataaactcttaaaaaatcggtattgaaagtacgcaccaccaaataaagcgtcgaggtcgggcatgtcttcgtggcgactcaaaatgagcaagtgttcaggttccagcagcgtccagactctcgaggatctgcgtaatcgtcttcgctccgaggccttgggcaacgattgccaagagccccgcgacgacctcttccccagcggcgaggagtgtctggacatcgacgggccctgcccttgcgatgaggcggagcaggagatcgaccaggagcagttgcccgtgcccgaaaccgtgcccgaaccgccggccaagactcctaagcgccgaccagtgaagaaggataaggcagataaggcagataaggacaagtcgaccagaggcgcaaagaaaccgtgccttcggacgacaaggatgacgagctcaagagcaacgacgtcgacaacaacgaagagtccggcgacacagacggcggcgagcgcccgaagccccagcgacatcgacaacgtggacgaaatggacgactccgacctcatggtggcgttctccaccatcctcgcagacttcaaggaccttacccaacgagtgaaagctctttcgtccgtgctcacggacgtgcaggcggccggcatacgcaggagcttctcgacgctcggcaaggctctgacggaggcggcccacatcgccaacaccggatctaagccagtcactgcgcctcgcaagaagaaggccgccgcctgcaagaagtaggcgcactaaatagcgaggctcggtatgcgggcgctgcacctgtcagacggcaaacttttttttgacaaggagctgacgcagccggtccccgacgacaaccccgcgtacgctgtccttgcgaagatccggatcccaccgcacctctcggatgtggtcgtgtacgagcaggacctcgagtctgcgcagcagggcctcatcttcgtcgggcgcgacgccaagggccgaaagcagtacttctacgggcgcggacacgtggagcggcgcacggccgtccgcaacgccgtgttcgtgcgcgtgcaccgcgtcatgaacaagataaacgccttcatcgacgaccacctcgcctccggcagcgaggccgaggcgcagatggccgccttcctgctcatggagacgagcttcttcatccgcgtcggcaagacgcgctacgagcgcgagagcggcaccgtgggcatgctcacgctgcgcaacaagcacctcgccgaggccgagggcggtgaggagatccgcgtcgccttcgtgggcaaggaccgagtcgcgcacgagtttgccgtgcgcgaggggcagcggctcttcgcggcgctgcgtcggctctgggacccgggcgcgcccgacaggctgctgttcgaccggctgagcgagcgccgcgtgtacaccttcatgcgacgcttcggcatccgcgtcaaggacctgcgcacctacggcgtgaactacaccttcctgtacaacttctggtccaacgtgcgctcgctggagccgcgtccctccgtgaagtcgctcatctgcacctccgtgcggcagaccgccgagacggtggggcacacgccctcgatctcgcgcagcgcctacatggccaccgcggtgctcgagctcgtcagggacggcgcgttcctggacagagtcgccgccaccgacacgctcgacgacttcgtggacatcgtcgtggactatgtaaataactctgagcaggtaaatggatgaggcgctgcgcgtggcggcgcgcgtcgtggacgggctccggccgctggacgtggccgtgtgtctcgcgcagctgcgcggagccgcgcccgagcgccgcttcccggcgctcgacgagtgctccggcgaggccttcctggactttgagttcgccggcggggacgtggcgtcgcggtacc

SEQ ID NO: 5 - DNA, 4020 bp, Orf virus strain NZ-2
agtgagcgggcgggcgataaaaataatcaattgactgattcgctcgtgagcgagcgaagggcggcggacaagggcgcgggatgctggtctaatctactaaggccgattacaaaaacggatgggagaccgggagggagagggtcacagctccgagcggtgcatccgcgccagctggcggcgccactcggccgcgggccgcggccgcccaggccgcgttgtagccgcccgccaccgcgacgcaggtcagctcgaactccggcccgagcgcgcgcacgtcgtagatgtgcacggtcgcgacgttcagcagcagcgcgcccttgcgcagcgaggcgaaggtcgcgtgcatgccggcggcgagcgggtacaccggcgagagcgtcccgccgccgtgcgcgaccaccgccatgtgccgcccgtcgccgccgacggtcaggcaggtcaccgaggcggagccgttcgcgcggacggggcccgcctccacgagggcggccggcagcggcggcgccgcggggcggaagagcccgccgaggaggaagcccagcgccaccagcgcgagcgcgccgagcagcctgcgcggcgaggggtgcatgcttgttggctgcggtgttggcgtctggcgggtggaaggcgggtgtggggtgtccgcggctgcggcgggagggtctcgactgctaggcggtcctttttcactttgctccgtggcgcctggtccggggcaagggctgcggcgggcgcccgggcgggcgggccgctaccccgccgcgcggcccgcggccgccagcgccgcggccagccgccgccaccgcgggcccgccgcgcgcagcagccccgccgccgagcgccccgcgccgccgcgggcgcgcgccgaggccgcggccccgcgccgcgccagcagcagcggcagccgcgcgtccagcgggccgccgcggcgcagcgccgcgcgcagcagcgccgccagcggcagccgccccgccgcgtccgggcccgccgcgcgcgcgcccgccgccagcagcgcgcgcaccagcgccggcgagggcgccgcgcgcggaccaggtgctccacgagcagggtggtgagcaaggattctcgagaagtaggagtcatgtgtgacgacaaggagagacgttatattaggcgcgtcctacttcactttgaagatggtgtaaagtgttaaaacttgaacaccgttcactccaccactgccgttaccgtgtcctgccccaaaagcaaccacagtgctttttccaccacctgttccaaatccgttccaaaagctcccatccattgttgttagaactttcagatgtttctctaggttgtttagttccactgcaagtttcgaccattatcgttactggacatgctgttggtaatgagtttaataaccaatcataaaaatagttataatttgttataaagctaataaagtagcaaacactttaatgttatattttgcctaaccctccgttaacaccaccattaacaccaccacttaagcttttactaccaccactaccacctccaacacacattcttttctctaaaggtccccaaattccacctcctgaacttggacgttttacagcacctccgggtgtacttgcgtaccctttagaagttccactgtgactgtagatatgatactgtccttctccaggcatgattaaagtgtgttgtaattagtgttatctacgcaactgtgcgagactctcgaataaaaagaagctacattttacaattttgattagctgatgtaccacgctgtatcgcggccaccacaagcacccgatccagtagaaccaaatccagagtcgccgcggtcagtgttgtccaagcagttaacctcttgaactgctgggcacgatatgcgttcgcatattagctgagctatcctgtctcccttcttaacctcaaagtcactgtttccaaagttaaacagcaccactccgacgttgcctcggtagtcttcgtcgatcacgccagcgcccacgtcgataaagtgtttgactgcaaggccagaacgtggtgctatgcgtccgtagcaaccagaagggggctttatcagaaggtcagtaaatactacgcgactgcaatgcgaagggatgacacagtcgtatgcactacataggtctaatcctgcggcaccaggagatcctctggctggtatagtggcgttttggctgaggcgaacaacctgaagagtttccgtgtggcagaactccatggctagggtggcgagcggccgatcgactacggggtgtacaatttacactttctccagaaaaatcaggggcgggtcagcatggcgcggcgcaagtccagcagcgagtcgtacgacaggaagcacaggatggaggtcacgatctccggcggcagggcgcacgggcacatgaggccggcgatctgctcggccagcgagacgcgcagccgcatcatgcagatcttgccgaagagcgccgtcccgtagatggggaactcggccgcgcgctccaaaaaggcgttctgcacgaagagcgccttcgcgtcgtccgacgcgcgcagcacgtccaacagcgtcgcgtccgtgtggcagcgcaccgcgcgcatgcttgcgatctcctgctcgcacgcgcggatcacggtcgcgtagtccgccagcgcgcgctccgccagcatgcgcgcgccctcgccgcgcagcgccagctcctgcacgcacagcagcgcggcctccgagcgtcggaacacgtggccccattgctcggatgtgatcagcgcgcgcgcgagcagctccgtcggcggccggcgcgcgagcacggccgccgtcgcgcgcacgttgttgcggcgcagcatctccgaaaccgcgcataggcccgaggccgacatgtgctcgagctccgcgcccatgcgcaccagccggcagcaggcgccgtggctgaacaccgccgcgcggtgcagcgcggtctgcaggttgttgttgcgcaggttcaggtccagcccgcgctcgagcacgaagtccacgacgccgcgctcgcagctcccgtaggtcgccatgtagtgcagcatggtgttcccacacgcgtctacggcggccgggtccacgcctaggcccgtgagcgtgcgcaccatgccctcggagatcttggccgtgcgcgcgaggtggtgcagcgttgtgcgcccgtacgcgtccacgacgcacgcgtccgcgcccgcgcgcagcatcatgtccacgagcgcggcggagacgccgccggagcacagcagcgccgccagcggcgtcaagccgttgcagtcgcaggcgtttgggttcgcgccgcgctcgagcagcagccgcagcacgtcctcgcggatccactggttcttggcgtacacgtgcagcggcgttacgccgtaggtgttgccctcgttcacgcgcgcgcccgcgtccagcagcagccgcgcgacctcgagctcggcgccgtcggggccgcagaaagccaggaaggaggagagcacgctgtcgcagacaacgacgctggcgtcgcagaccacgtccgcgcccgcctccagcatgagcgcgaccacctccggccgcacgccgtcgtactgcacgtaggcgtgcagcggcgtgcggccgcaggagtccttggctttcacgtccgcaccggcctccagcagcacgcgcacgatctccgcgcactgctcgtgccgcgcgaagtgcacgcagaggtgcagcggcgtgcgcccgtgctcgccgcggaagttcacgtctgcgtcggtggctacgagcgcgcggaccgtttcgaggtccacctgcccggactccaggtagcggaagagcaggtccgcgtgcgggaccacgacggactcccgcgagagcatggcggcgtttacaaatattgaaatcttttttcactcatctttatggcgctgaacgcgcaataagggtgagagtaaaaaacttctacaaaaagcgtacaaaaggtacaaaaggtaaaaaaggcggggcggggacgggctggggtgctgcgagctgaattggcctctacacagggacgccctcgc

SEQ ID NO: 6 - DNA, 34 bp, Orf virus strain NZ-2
gaaagtgtaaattgtacaccccgtagtcgatcgg

SEQ ID NO: 7 - DNA, 34 bp, Orf virus strain NZ-2
aaaattgtaaaatgtagcttctttttattcgaga

SEQ ID NO: 8 - DNA, 34 bp, Orf virus strain NZ-2
gcaaagtgaaaaaggaccgcctagcagtcgagac

SEQ ID NO: 9 - DNA, 34 bp, Orf virus strain NZ-2
gatgagtgaaaaaagatttcaatatttgtaaacg

SEQ ID NO: 10 - DNA, 34 bp, Orf virus strain NZ-2
aataactgataaaatatgtttttttggttttggt

SEQ ID NO: 11 - DNA, 34 bp, Orf virus strain NZ-2
ataaattaaaattaaagcgcggaggctcgaacgc

SEQ ID NO: 12 - DNA, 34 bp, Orf virus strain NZ-2
aattattgaaaatgtaggcgcgataaacacacgt

SEQ ID NO: 13 - DNA, 49 bp, Orf virus strain NZ-2
gccgccataaaagagttgtatatgattaattttaataactaaatggatc

SEQ ID NO: 14 - DNA, 49 bp, Orf virus strain NZ-2
tttcagttttgagacggtctcgcgcgcccttgccgtccttaaatggatt

SEQ ID NO: 15 - DNA, 49 bp, Orf virus strain NZ-2
actcttaaaaaatcggtattgaaagtacgcaccaccaaataaagcgtcg

SEQ ID NO: 16 - DNA, 49 bp, Orf virus strain NZ-2
cgcaagaagaaggccgccgcctgcaagaagtaggcgcactaaatagcga

SEQ ID NO: 17 - DNA, 49 bp, Orf virus strain NZ-2
aagactttccctgaagccctattatttttgtgagataaataaatgtggc

SEQ ID NO: 18 - DNA, 49 bp, Orf virus strain NZ-2
ggagctgcgcgagctccgcgccaacgaataattctgcacataaaagatg

SEQ ID NO: 19 - DNA, 49 bp, Orf virus strain NZ-2
atattagataaccgctgtgttgccgtctgtaattatttaattataaatg

SEQ ID NO: 20 - DNA, 49 bp, Orf virus strain NZ-2
gtaataaggattaacatacgaagttgggataattatttaattataaatg

SEQ ID NO: 21 - DNA, 49 bp, Orf virus strain NZ-2
ttcgtggacatcgtcgtggactatgtaaataactctgagcaggtaaatg

SEQ ID NO: 22 - DNA, 21 bp, Artificial Sequence
  Description of Artificial Sequence Made in lab.
gatcccgctcgagaacttcaa

SEQ ID NO: 23 - DNA, 39 bp, Artificial Sequence
  Description of Artificial Sequence Made in lab.
gtcagatctatgcataaaaatttcgcatcagtcgagata

SEQ ID NO: 24 - DNA, 40 bp, Artificial Sequence
  Description of Artificial Sequence Made in lab.
gacatgcatcagtgccatggaattctcgcgactttctagc

SEQ ID NO: 25 - DNA, 26 bp, Artificial Sequence
  Description of Artificial Sequence Made in lab.
gacggatccgtataatggaaagattc

SEQ ID NO: 26 - DNA, 117 bp, Artificial Sequence
  Description of Artificial Sequence Made in lab.
gactgatgcgaaacgcgcggcggcgccgcgacttagcttatctcgactgatgcgaaatttttatgcatcagtgccatggaattctcgcgactttctagcttctcagactgatgctac